(19) United States
(12) Patent Application Publication
(10) Pub. No.: US 2012/0263646 A1
(43) Pub. Date: Oct. 18, 2012

(54) IMAGING AGENTS AND THEIR USE FOR THE DIAGNOSTIC IN VIVO OF NEURODEGENERATIVE DISEASES, NOTABLY ALZHEIMER'S DISEASE AND DERIVATIVE DISEASES

(75) Inventors: Sarah Catoen, Livry Gargan (FR); Thierry Gautheret, Bois Le Roi (FR)

(73) Assignee: GUERBET, Villepinte (FR)

(21) Appl. No.: 13/501,838

(22) PCT Filed: Oct. 15, 2010
(86) PCT No.: PCT/EP10/65526

§ 371 Date: Jun. 25, 2012

Related U.S. Application Data
(60) Provisional application No. 61/251,916, filed on Oct. 15, 2009.

(51) Int. Cl. A61K 51/04; A61P 25/28; C07D 417/04

(52) U.S. Cl. 424/1.89; 546/270.1

(57) ABSTRACT

Disclosed are new imaging agents and their use for the in vivo diagnostic of neurodegenerative diseases, notably Alzheimer's disease and related diseases.

The present invention relates to new imaging agents and their use for the in vivo diagnostic of neurodegenerative diseases, notably Alzheimer's disease and related diseases.

Alzheimer's disease (AD) is the most common form of dementia in elderly. Nowadays, several millions of patients suffer from AD and this number is expected to increase exponentially with the lengthening mean life span. AD is a slow progressive neurodegenerative brain disorder characterized by irreversible memory loss, deterioration of cognitive function along with behavioural symptoms, language impairment and disorientation.

Post-mortem examination of AD brain sections reveals abundant extracellular senile plaques (SPs) and numerous intraneuronal neurofibrillary tangles (NFTs); both of them along with activated microglia and reactive astrocytes have been commonly accepted as the hallmark of AD [1,2].

SPs originate from insoluble neurotoxic deposits of Ab40 and Ab42 peptides on neurons (‘Ab-plaque’ or ‘amyloid plaque’), resulting from cleavage of the amyloid precursor protein (APP) by specific proteases whereas NFTs are formed by filaments of highly phosphorylated tau proteins.

Clinical diagnosis of AD based on neurological observations by neuropsychological tests such as Mini-Mental State Examination is often difficult, unreliable and only yields indirect information. Since the deposition of Ab plaques is an early event in the development of AD, a validated biomarker of Ab deposition in the brain would be very helpful for identifying and following individuals at risk for AD and for assisting the evaluation of new anti-amyloid therapies currently under development. Therefore, detection and quantitative evaluation of Ab plaques in the brain with non-invasive techniques such as positron emission tomography (PET) and single photon emission computed tomography (SPECT) is useful for presymptomatic identification of patients and monitoring the effectiveness of novel treatments.

To allow non-invasive in vivo diagnosis of AD, radiolabelled derivatives of a number of immunohistochemical dyes (Congo red, chrysamine G, thioflavin S, or thioflavin T) have been tested and reported [4]. X-34 derivatives consisting of only half the X-34 molecule without carboxylic groups, the so-called stilbenes (SBs), showed promising results [8]. The reported carbon-11 labelled SB-13 has already been tested in subjects with mild to moderate AD and healthy controls, showing a high accumulation in the frontal and temporoparietal cortex of patients with AD but not in age-matched control subjects [9]. Barrio and co-workers, on the other hand, developed [18F]FDDNP, a naphthalene derivative that binds to amyloid at a different binding site and also to the NFTs [10]. It was reported that non-ionic analogues of Thioflavin T, an ionic imaging dye, penetrate the blood-brain barrier (BBB) and show high affinity for Ab-plaque. The most promising of all reported compounds seems to be the carbon-11 labelled 6-OH-BTA or 6-hydroxy-2-(40-N-[11C]methylaminophenyl)-1,3-benzothiazole, also known as Pittsburgh Compound-B (PIB). PIB has already been tested intensively in several clinical studies showing clear differences between AD, mild cognitive impairment (MCI) and control subjects [12]. However, this agent is labelled with short-lived carbon-11 (t1/2=20.39 min), which limits its availability to centres equipped with a cyclotron. This limitation may be overcome by introducing a fluorine-18 label which has a longer half-life (t1/2=109.8 min) and thus allows to provide a positron emission tomography (PET) tracer that is useful for a widespread clinical application: early clinical evaluation of fluorine-18 labelled derivatives (BAY94-9172, Av-45, GE-067) is also ongoing [13-15].

Many of the known amyloid targeting compounds are polycyclic compounds that contain a first part A consisting in a 6 or 5 membered cycle fused with or substituted by an other 6 or 5 membered cycle, linked to a second part B that is typically an aromatic mono or polycyclic group, leading to compounds (I) illustrated in the following formula:

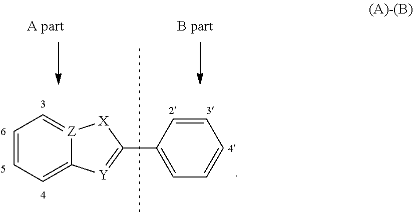

Known scaffolds comprising heteroatoms N, O or S at the X, Y, Z positions are notably benzothiazoles, benzofuranes, benzothiophenes, aminopyridines also described notably in patent documents WO2007/033080, WO 2007/124345, WO2007/047204, WO2008/134618, WO2008/118122, WO 2007/086800, WO 2008/0657875, WO 2007/011834, WO 2003/068269, WO 2008/124812, WO 2008/073350, WO 2007/045593, WO 2007/126733.

Known compounds of the prior art are for instance the following.

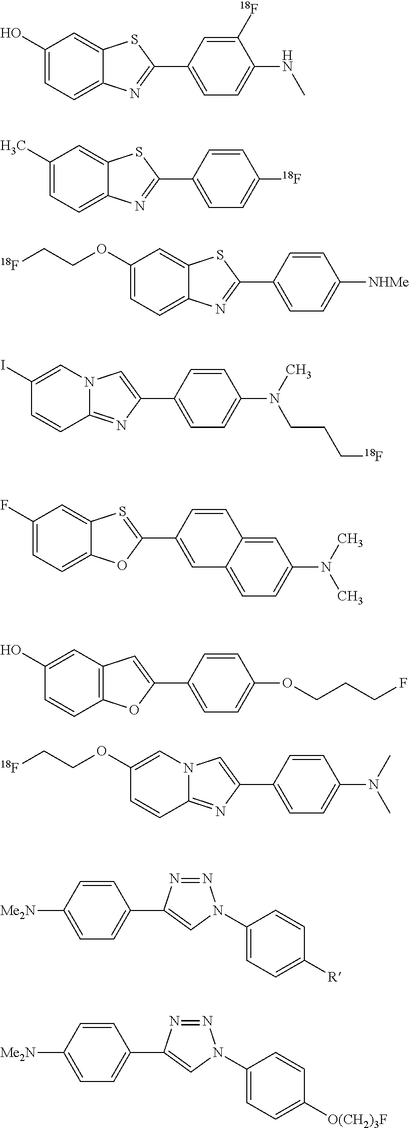

According to the knowledge of the applicant, when the aromatic cycle B of the prior art is a 6 membered cycle, the atom of the cycle B at the position 4′ is a C atom which is substituted by a Y group that is frequently a group NR1R2 (R1 and R2 being notably a H atom or an alkyl group). The Y groups are described in the literature as stabilizing groups and/or as groups that are useful for the biological targeting affinity. Recent documents WO 2007/086800 or WO2007/126733 describe some 6 membered B heterocycles that contain N atoms replacing some of the C atoms. However, the N atoms are described in positions 2′, 3′, 5′, 6′, but not at the 4′ position. Further, B groups disclosed in the prior art are consistently chosen from 6 membered ring structures.

Despite the high number of compounds investigated in the prior art, a few of which being at the clinical human stage, there is still a need for novel and efficient compounds, in particular for the early diagnostic of Alzheimer's disease. Among the huge number of possibilities of compounds derivating from those known in the art, the applicant has now worked on specific selected new compounds useful for Alzheimer's disease imaging which have a new B cycle, and lead to promising biological efficiency.

According to a first aspect the invention concerns compound of formula (I):

(A)-(CH₂)ₙ—(B),

wherein:
n represents 0 or 1 to 6,
(A) represents a 5 or 6 membered, saturated, unsaturated or aromatic cycle A1 comprising none to 5 heteroatoms chosen from N, S or O, which is:
either fused with a mono or fused bicyclic ring structure A2, said A2 being chosen from 5 to 10 membered aryl, cycloalkyl or saturated, unsaturated or aromatic heterocycle groups;
or substituted by a 5 or 6 membered ring structure A3 chosen from aryl, cycloalkyl, or saturated, unsaturated or aromatic heterocycle,

wherein said A1, A2, A3 are optionally and independently substituted at each of their available positions with an identical or different R1 substituent,
B) represents a carbocyclic or heterocyclic ring structure chosen from (B1) or (B2):

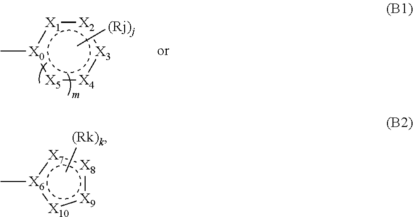

wherein

where

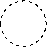

is saturated, unsaturated or aromatic as the case may be;

X₃ represent N;

X₀, X₁, X₂, X₄, X₅, X₆, X₇, X₈, X₉, X₁₀ represent N, C, S or O, preferably N or C,

m represent 0 or 1,

j represent an integer from 0 to 4 when m is 0 and from 0 to 5 when m is 1,

k represent an integer from 0 to 4;

for each (i), (j) and (k), each R1, Rj and Rk may be identical or different and are independently chosen from:
a)—H where i, j or k is 0,
   a linear, branched or cyclic, saturated or unsaturated aliphatic group, optionally substituted by one or more of Halogen, CN, NO₂, CHalo₃, COR₃, COOR₃, CONR₃R₄, NCOR₃, NHSO₂R₃, SR₃, SOR₃, SO₂R₃, OR₃ or NR₃R₄, wherein R₃ and R₄ represents independently H or a linear, branched or cyclic alkyl group optionally substituted by one or more of Halogen;
   a Halogene, CN, NO₂, CHal₃, OR₁ or NR₁R₂, COR₁, COOR₂, CONR₁R₂, NCOR₁, NHSO₂R₁, SR₁, SOR₁ or SO₂R₁ groups, wherein R₁ and R₂ represent independently H or a linear, branched or cyclic alkyl group optionally substituted by one or more of Halogen or R₁ and R₂ form together with the N atom to which they are attached a N-containing heterocycle;
      or
b)—a linear, branched or cyclic, saturated or unsaturated aliphatic group, substituted by one or more of leaving group and further optionally substituted by a Halogen, CN, NO₂, CHalo₃, COR₃, COOR₃, CONR₃R₄, NCOR₃, NHSO₂R₃, SR₃, SOR₃, SO₂R₃, OR₃ or NR₃R₄, wherein R₃ and R₄ represents independently H or a linear, branched or cyclic alkyl group optionally substituted by one or more of Halogen;
   a leaving group;
   or NR₁R₂, COR₁, COOR₂, CONR₁R₂, NCOR₁, NHSO₂R₁, SR₁, SOR₁ or SO₂R₁ groups, wherein R₁ or R₂ represent a linear, branched or cyclic alkyl group substituted by one or more of leaving group;
      or
c)—a linear, branched or cyclic, saturated or unsaturated aliphatic group, substituted by one or more of Halogen and further optionally substituted by one or more of CN, NO₂, CHalo₃, COR₃, COOR₃, CONR₃R₄, NCOR₃, NHSO₂R₃, SR₃, SOR₃, SO₂R₃, OR₃ or NR₃R₄, wherein R₃ and R₄ represents independently H or a linear, branched or cyclic alkyl group optionally substituted by one or more of Halogen;
   a Halogene, CHal₃,
   OR₁ or NR₁R₂, COR₁, COOR₂, CONR₁R₂, NCOR₁, NHSO₂R₁, SR₁, SOR₁ or SO₂R₁ groups, wherein R₁ or R₂ represent a linear, branched or cyclic alkyl group substituted by one or more of Halogen;
      or
d)—a linear, branched or cyclic, saturated or unsaturated aliphatic group, substituted by one or more of R₁₀ and further optionally substituted by one or more of Halogen, CN, NO₂, CHalo₃, COR₃, COOR₃, CONR₃R₄, NCOR₃, NHSO₂R₃, SR₃, SOR₃, SO₂R₃, OR₃ or NR₃R₄, wherein R₃ and R₄ represents independently H or a linear, branched or cyclic alkyl group optionally substituted by one or more of Halogen;
   a R₁₀ group;
   OR₁ or NR₁R₂, COR₁, COOR₂, CONR₁R₂, NCOR₁, NHSO₂R₁, SR₁, SOR₁ or SO₂R₁ groups, wherein R₁ or R₂ represent a linear, branched or cyclic alkyl group substituted by one or more of R₁₀;

wherein R₁₀ is a radionuclide, in particular selected from the group consisting of ¹²⁰I, ¹²³I, ¹²⁴I, ¹²⁵I, ¹³¹I ⁷⁶Br, ⁷⁵Br, ¹⁸F, ¹⁹F, ¹¹C, ¹³C, ¹⁴C, ⁹⁹Tc and ³H, preferably fluoro ¹⁸F;

and their pharmaceutically acceptable salts.

Preferably

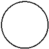

in A and B rings are unsaturated or aromatic. Even preferably

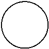

in A and B are aromatic, leading to a higher constraint in the tridimensional conformation.

Preferably, at least one of Ri, Rj or Rk is chosen from groups b), c) or d).

Advantageously, the five membered cycles B2 contain one or two Rk group(s) that is(are) a fluoro containing group.

Particularly:
n is 0 or n is 1 (preferably n is 0)
A1 comprises 1 to 5 heteroatoms;
j is 1 or 2;
k is 1 or 2.

Said leaving group is preferably chosen from:
an halogen, preferably I, Cl or Br,
NO₂,
OSO₂R₉, wherein R₉ represents a linear, branched or cyclic alkyl or a phenyl optionally substituted by a linear alkyl, such as a mesylate or a tosylate,
Triflate, and/or
CN.

According to an aspect of the invention, at least one of Ri, Rj or Rk is chosen from a leaving group containing group b) or an halogen containing group c), the remaining of Ri, Rj or Rk being chosen from groups a). Said corresponding compounds are thus non labelled and are herein referred to as precursors.

Said compounds are useful as for preparing corresponding agents of formula (I) where at least one of Ri, Rj or Rk is chosen from radionuclide containing groups d).

According to another aspect of the invention, at least one of Ri, Rj or Rk is chosen from a R10 containing group d). Corresponding compounds comprising a d) group are thus labelled and are thus useful as contrast agents; they are herein called “labelled compounds”. They may be prepared extemporaneously from said precursors in the presence of suitable reagents generally used for introducing said radionuclide.

Said compounds are useful for in vivo imaging of amyloid deposits.

Among the compounds of formula (I), particular compounds are those wherein:

1) A is selected from:
1.1) a fused A1-A2 cycle of formula

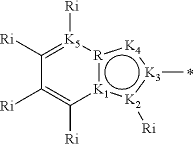

wherein the presence of the R1 linked to K₂ is optional;
and wherein:
K1 is C or N;
K2 is C or N or O or S;
K3 is C or N;
K4 is C or N or O or S;
K5 is C or N; and
R is C or N;
and wherein preferably:
K2 is S, K4 is N, the remaining K1, K3, K5 are C; or
K2 is O, K4 is N, the remaining K1, K3, K5 are C; or
K1 and K4 are N, the remaining K2, K3, K5 are C; or
K2, K3, K4 are N, the remaining K1, K5 are C; or
K2 is O, K4 and K5 are N, the remaining K1, K3 are C; or
K1, K4, K5 are N, the remaining K2, K3 are C; or
K2 is O or N, K4 is C, the remaining K1, K3, K5 are C; or
K2 is N, K4 is N, the remaining K1, K3, K5 are C; or
K2 and R are N, K5 is C, the remaining K1, K3, K4 are C; or
K2 is N, K4 is O or S, K5 is N or C, the remaining K1, K3 are C; or
K2 is N, K4 is O or S, the remaining K1, K3, K5 are C; or
K2 is N, K4 is O, the remaining K1, K3, K5 are C.
1.2) a A1 cycle substituted by a A3 ring, advantageously of formula A1-A3

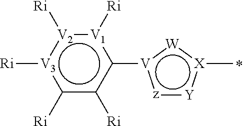

wherein V1, V2, V3, identical or different, are C or N;

X and V, identical or different, are C or N; and

W, Y, Z, identical or different, are C or N or O or S; where

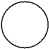

is saturated, unsaturated or aromatic as the case may be; or
1.3) a A1-A3 of formula

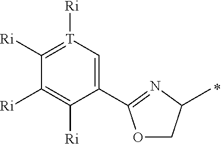

wherein T is C or N;

or
1.4) a A1-A2 fused cycle, of formula

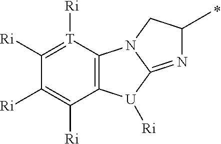

wherein U is C or O or S; and/or

T is C or N;

or
1.5) a A1-A2 fused cycle of formula

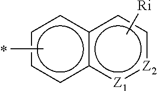

wherein Z1 and Z2, identical or different, are C or N;

or
1.6) a A1-A2 fused cycle of formula

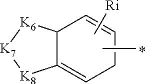

wherein K6, K7, K8, identical or different, are C or N,

wherein the (*) represents the place of the link (carried by ring cycle A1) and linking the cycle A1 to the —(CH₂)ₙ group of —(CH₂)ₙ—(B);

and/or

2) B is chosen among a 6 membered cycle (B1) or a 5 membered cycle (B2) of the respective formula:

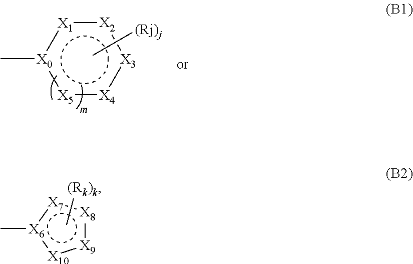

wherein:
2.1) m is 0 or 1; and/or
2.2) X3 is N; and/or
2.3) X₀, X₁, X₂, X₄, X₅, X₆, X₇, X₈, X₉, X₁₀ are independently selected from N or C;
   and wherein
   each of R1, Rj and Rk identical or different are independently selected from:
H, fluoro, chloro, iodo, bromo, C1-5 alkyl, methyl, hydroxy, methoxy, hydroxyalkyl, C1-5 haloalkyl, C1-3 alkyleneO C1-3 alkyl, C1-3 alkyleneO C1-3 haloalkyl, C1-3 alkyleneNH₂, C1-3 alkyleneNH C1-3 alkyl, C1-3 alkyleneN(C1-3 alkyl)₂, C1-3 alkyleneNH C1-3 haloalkyl, C1-3 alkyleneN(C1-3 haloalkyl)₂, C1-3 alkyleneN(C1-3 alkyl)C1-3 haloalkyl, C1-5 haloalkoxy, C1-5 alkylthio, C1-5 haloalkylthio, amino, NH C1-3 alkyl, C1-3 haloalkyl, N(C1-3 alkyl)₂, N(C1-3 alkyl)C1-3 haloalkyl, NH(CO)C1-3 alkyl, NH(CO)C1-3 haloalkyl, NH(CO)C1-3 alkoxy, NH(CO)C1-3 haloalkoxy, NHSO₂C1-3 alkyl, NHSO₂C1-3 haloalkyl, (CO)C1-3 alkyl, (CO)C1-3 haloalkyl, (CO)C1-3 alkoxy, (CO)C1-3 haloalkoxy, (CO)NH₂, hydroxy, metoxy, C1-5 hydroxyalkyl, (CO)NH C1-3 alkyl, (CO)NH C1-3 haloalkyl, (CO)N(C1-3 alkyl)₂, (CO)N(C1-3 alkyl)C1-3 haloalkyl, (CO)N(C4-6 alkylene), (CO)N(C4-6 haloalkylene), cyano, SO₂NH C1-3 haloalkyl, nitro and SO₂NH₂;
   and more particularly chosen from:
H, fluoro, C1-5 alkyl, methyl, hydroxy, methoxy, hydroxyalkyl, C1-5 fluoroalkyl, C1-3 alkyleneO C1-3 alkyl, C1-3 alkyleneO C1-3 fluoroalkyl, C1-3 alkyleneNH₂, C1-3 alkyleneNH C1-3 alkyl, C1-3 alkyleneN(C1-3 alkyl)₂, C1-3 alkyleneNH C1-3 fluoroalkyl, C1-3 alkyleneN(C1-3 fluoroalkyl)₂, C1-3 alkyleneN(C1-3 alkyl)C1-3 fluoroalkyl, C1-5 fluoroalkoxy, C1-5 alkylthio, C1-5 fluoroalkylthio, amino, NH C1-3 alkyl, C1-3 fluoroalkyl, N(C1-3 alkyl)₂, N(C1-3 alkyl)C1-3 fluoroalkyl, NH(CO)C1-3 alkyl, NH(CO)C1-3 fluoroalkyl, NH(CO)C1-3 alkoxy, NH(CO)C1-3 fluoroalkoxy, NHSO₂C1-3 alkyl, NHSO₂C1-3 fluoroalkyl, (CO)C1-3 alkyl, (CO)C1-3 fluoroalkyl, (CO)C1-3 alkoxy, (CO)C1-3 fluoroalkoxy, (CO)NH₂, hydroxy, metoxy, C1-5 hydroxyalkyl, (CO)NH C1-3 alkyl, (CO)NH C1-3 fluoroalkyl, (CO)N(C1-3 alkyl)₂, (CO)N(C1-3 alkyl)C1-3 fluoroalkyl, (CO)N(C4-6 alkylene), (CO)N(C4-6 fluoroalkylene), cyano, SO₂NH C1-3 fluoroalkyl, nitro and SO₂NH₂; —N(R11)₂, and OR11 where R11 is H or C1-3 alkyl.

As used herein, “halo”, “chloro”, “fluoro”, “iodo” and “bromo” are meant to encompass the radioisotopes of Cl, F, I and Br atoms.

Advantageously, the compounds (I) are such that at least one of Ri, Rj, Rk comprise at least one detectable label selected in the group consisting of labelled halogen, such as ¹³¹I, ¹²³I, ¹²⁴I, ¹²⁵I, ⁷⁶Br, ⁷⁵Br, ¹⁸F, ¹⁹F, ¹¹C, ¹³C, ¹⁴C, ⁹⁹Tc and ³H, preferably fluoro ¹⁸F.

Advantageously, the compounds (I) are such that at least one Ri, Rj, Rk, preferably at least one Rk, is fluoro, chloro, bromo, iodo or a fluoro containing group chosen from: C1-5 fluoroalkyl, C1-3 alkyleneOC1-3 fluoroalkyl, C1-3 alkyleneNHC1-3 fluoroalkyl, C1-3 alkyleneN(C1-3 fluoroalkyl)₂, C1-3 alkyleneN(C1-3 alkyl)C1-3 fluoroalkyl, C1-5 fluoroalkoxy, C1-5 fluoroalkylthio, NHC1-3 fluoroalkyl, N(C1-3 alkyl)C1-3 fluoroalkyl, NH(CO)C1-3 fluoroalkyl, NH(CO)C1-3 fluoroalkoxy, NHSO₂C1-3 fluoroalkyl, (CO)C1-3 fluoroalkyl, (CO)C1-3 fluoroalkoxy, (CO)NHC1-3 fluoroalkyl, (CO)N(C1-3 alkyl)C1-3 fluoroalkyl, (CO)N(C4-6 fluoroalkylene), SO₂NHC1-3 fluoroalkyl.

Preferably, in compounds of formula (I), Ri, Rj, Rk, are chosen from H, fluoro, chloro, bromo and iodo, at least one of Ri, Rj, Rk being preferably a detectable label selected in the group consisting of labelled halogen such as ¹³¹I, ¹²³I, ¹²⁴I, ¹²⁵I, ⁷⁶Br, ⁷⁵Br, ¹⁸F and ¹⁹F.

Preferred A groups are selected from:
1.1) a fused A1-A2 cycle of formula

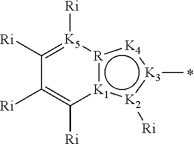

wherein the presence of the Ri linked to K2 is optional;
and wherein:
K1 is C or N;
K2 is C or N or O or S;
K3 is C or N;
K4 is C or N or O or S;
K5 is C or N; and
R is C or N; and
   the A ring being preferably imidazopyridine, benzofurane, benzothiazole or benzothiophene, and
1.2) a A1 cycle substituted by a A3 ring, advantageously of formula A1-A3

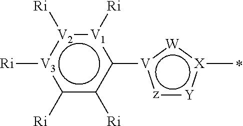

wherein V1, V2, V3, identical or different, are C or N;

X and V, identical or different, are C or N; and

W, Y, Z, identical or different, are C or N or O or S; where

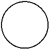

is aromatic.
preferably wherein A1 is thiophene or oxadiazole and A3 is phenyl or pyridine.

Preferred embodiments of the B1 and B2 cycles are chosen among those where X0 is C, X3 is N and:
one of X1, X2, X4, X5 is N, and the remaining are C; or
two of X1, X2, X4, X5 are N, and the remaining are C; or
X1 and X4 are N and the remaining X2, X5 are C; or
X2 and X5 are N, and the remaining X1, X4 are C; or
X1, X2, X4, X5 are C; or
X1 is N and the remaining X2, X4, X5 are C.

More preferably, B1 is

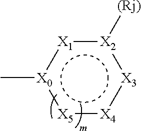

wherein X₃ is N, m=1 and Rj is chosen among:

chloro, fluoro, bromo, iodo, preferably fluoro; or

C1-5 fluoroalkyl, C1-3 alkyleneOC1-3 fluoroalkyl, C1-3 alkyleneNHC1-3 fluoroalkyl, C1-3 alkyleneN(C1-3 fluoroalkyl)₂, C1-3 alkyleneN(C1-3 alkyl)C1-3 fluoroalkyl, C1-5 fluoroalkoxy, C1-5 fluoroalkylthio, NHC1-3 fluoroalkyl, N(C1-3 alkyl)C1-3 fluoroalkyl, NH(CO)C1-3 fluoroalkyl, NH(CO)C1-3 fluoroalkoxy, NHSO2C1-3 fluoroalkyl, (CO)C1-3 fluoroalkyl, (CO)C1-3 fluoroalkoxy, (CO)NHC1-3 fluoroalkyl, (CO)N(C1-3 alkyl)C1-3 fluoroalkyl, (CO)N(C4-6 fluoroalkylene), SO₂NHC1-3 fluoroalkyl.

In a particularly preferred aspect, Rj is F.

In another embodiment, in (B1), m is 0 and at least one of X0 to X4 is N.

Preferably, the (B1) ring is a pyridine or a pyrrole ring.

The 5 membered cycle (B2) is preferably chosen from pyrroline, imidazoline, pyrazoline, pyrrole, pyrazole, imidazole, triazole, tetrazole, isoxazoline, oxazoline, oxazole, thiazole, oxathiazole, dioxazole, dithiazole, oxadiazole, thiadiazole, isoxazole, thiphene and furane being substituted by Rk groups as defined above.

The X6 to X10 of the 5 membered cycle (B2) are preferably chosen from N and C and the 5 membered cycle (B2) is preferably chosen from pyrroline, imidazoline, pyrazoline, pyrrole, pyrazole, imidazole, triazole and tetrazole, being substituted by Rk groups as defined above.

Preferably, the 5 membered cycle (B2) is an aromatic cycle and is chosen from pyrrole, imidazole and pyrazole, oxazole, thiazole, isoxazole, thophene and furane.

In a first embodiment, in the (B2) cycle, X6 is C and:
X7 and X8 are N and X9 and X10 are C (B2 is pyrazole); or
X8 is N, X10 is O and X7 and X9 are C (B2 is oxazole); or
X8 and X10 are N and X7 and X9 are C (B2 is imidazole); or
X10 is N and X7, X8 and X9 are C (B2 is pyrrole).

In a second embodiment, in the (B2) cycle, X6 is N. Preferably, X6 is N and:
X10 is N and X7, X8 and X9 are C (B2 is pyrazole); or
X7, X8, X9 and X10 are C (pyrrole); or
X9 is N and X7, X8 and X10 are C (imidazole); or
X9 and X10 are N and X7 and X8 are C (triazole); or
X8 and X9 are N and X7 and X10 are C (triazole).
Advantageously, X8, X7 and X6 are N, and X10 is C and X9 is C.

Advantageously (B2) are chosen from the following cycles:

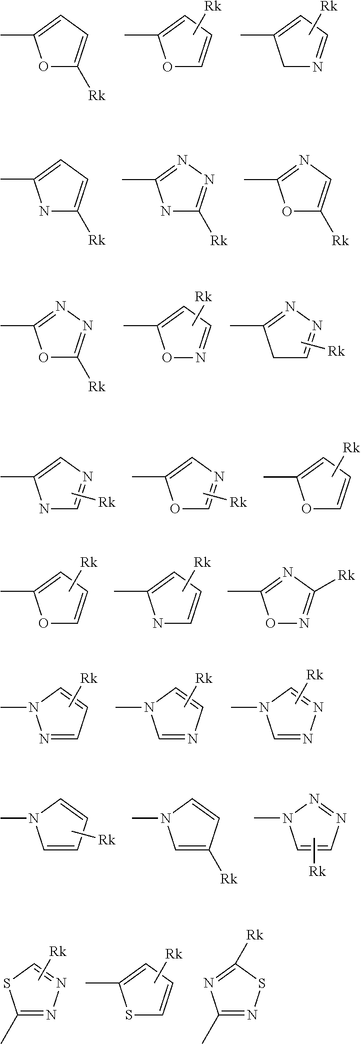

wherein Rk is as defined above.

Advantageously (B2) are chosen from the following cycles:

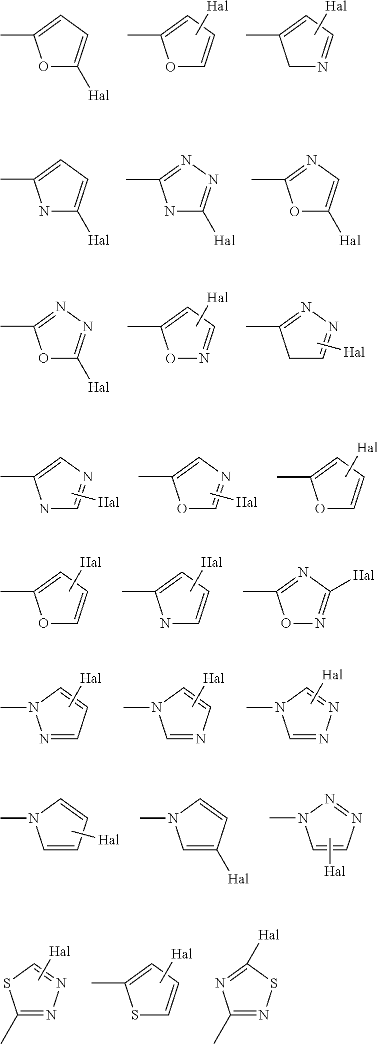

wherein Hal represents an halogen chosen from—chloro, fluoro, bromo iodo, preferably fluoro.

Advantageously (B2) are chosen from the following cycles:

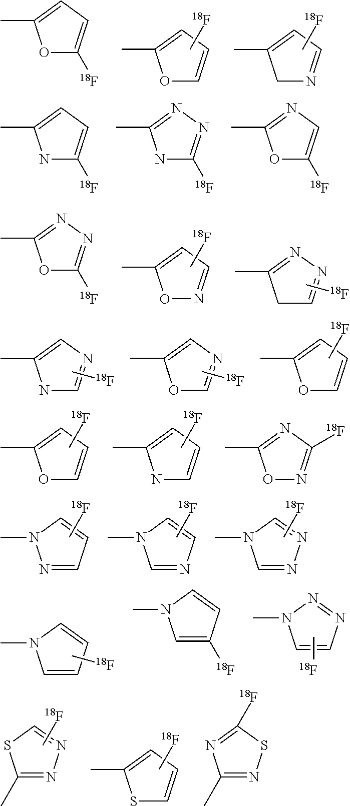

When the B2 cycles carry an unlabelled fluoro containing group, the B2 cycles are advantageously adapted for appropriate fluorination with a radiolabelled fluor atom as defined above.

In compounds A-B2, at least one of the Rk group is a fluoro containing group.

Advantageously in compounds A-B2, k is 1 or 2 and preferably k is 1.

Preferably one of Rk is:
chloro, fluoro, bromo iodo, preferably fluoro
   C1-5 fluoroalkyl, C1-3 alkyleneOC1-3 fluoroalkyl, C1-3 alkyleneNHC1-3 fluoroalkyl, C1-3 alkyleneN(C1-3 fluoroalkyl)₂, C1-3 alkyleneN(C1-3 alkyl)C1-3 fluoroalkyl, C1-5 fluoroalkoxy, C1-5 fluoroalkylthio, NHC1-3 fluoroalkyl, N(C1-3 alkyl)C1-3 fluoroalkyl, NH(CO)C1-3 fluoroalkyl, NH(CO)C1-3 fluoroalkoxy, NHSO₂C1-3 fluoroalkyl, (CO)C1-3 fluoroalkyl, (CO)C1-3 fluoroalkoxy, (CO)NHC1-3 fluoroalkyl, (CO)N(C1-3 alkyl)C1-3 fluoroalkyl, (CO)N(C4-6 fluoroalkylene), SO₂NHC1-3 fluoroalkyl.

The following table shows illustrative examples of compounds A-B obtained from any combination of A and B parts as defined above.

| | | Structure-provided A and B are |
|---|---|---|
| A | B | linked by a —(CH₂)ₙ-chain as defined above |
| A2- A1 | B1 | 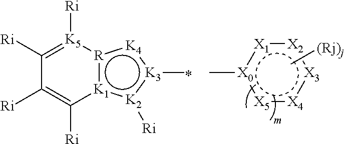 |
| A3- A1 | B1 | 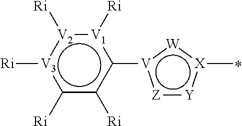 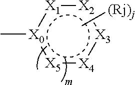 |
| A3- A1 | B1 | 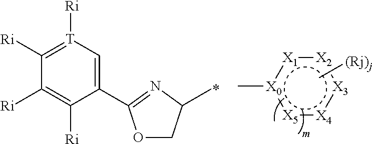 |
| A2- A1 | B1 | 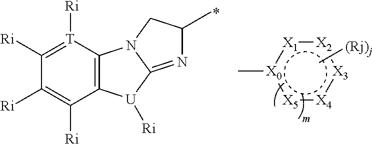 |
| A2- A1 | B1 | 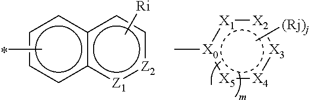 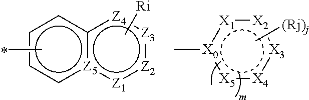 with Z1 to Z4 is CH or N, and Z5 is CH or N |
| A2- A1 | B1 | 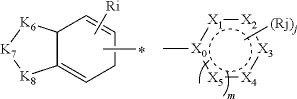 |
| A2- A1 | B2 | 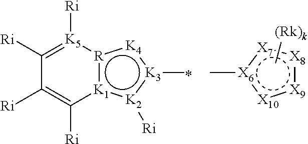 |
| A3- A1 | B2 | 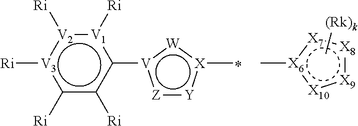 |
| A3- A1 | B2 | 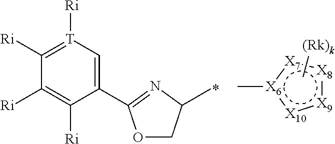 |
| A2- A1 | B2 | 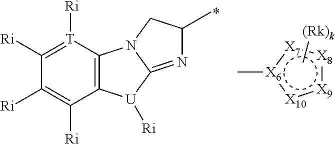 |
| A2- A1 | B2 | 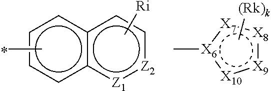 |
| A2- A1 | B2 | 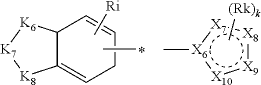 |
| A2- A1 | B2 | 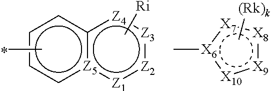 with Z1 to Z4 is CH or N, and Z5 is CH or N |

Preferred compounds of formula (I) are those wherein:

1) A is a fused A1-A2 cycle of formula

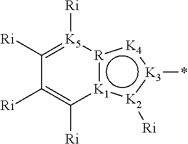

wherein the presence of the Ri linked to K2 is optional;
and wherein:
K1 is C or N;
K2 is C or N or O or S;
K3 is C or N;
K4 is C or N or O or S;
K5 is C or N; and
R is C or N; and

2) B is a 5 membered cycle (B2)

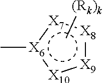

wherein X6 is N or C, advantageously X6 is C, and:
one to three of X7 to X10 is N, the remaining of X7 to X10 being C; or
one or two of X7 to X10 is O, the remaining of X7 to X10 being C or N; or
one or two of X7 to X10 is S, the remaining of X7 to X10 being C or N

(B2) being preferably chosen from the following cycles:

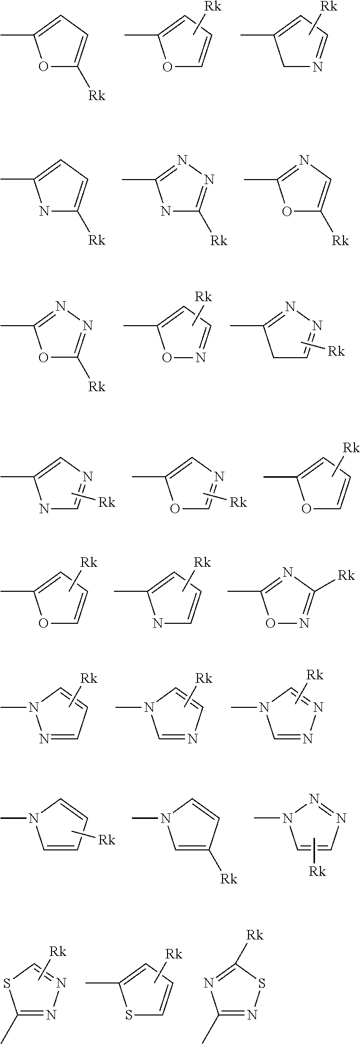

and wherein Ri and Rk are chosen among H, fluoro, chloro, bromo, iodo or a fluoro containing group chosen from: C1-5 fluoroalkyl, C1-3 alkyleneOC1-3 fluoroalkyl, C1-3 alkyleneNHC1-3 fluoroalkyl, C1-3 alkyleneN(C1-3 fluoroalkyl)₂, C1-3 alkyleneN(C1-3 alkyl)C1-3 fluoroalkyl, C1-5 fluoroalkoxy, C1-5 fluoroalkylthio, NHC1-3 fluoroalkyl, N(C1-3 alkyl)C1-3 fluoroalkyl, NH(CO)C1-3 fluoroalkyl, NH(CO)C1-3 fluoroalkoxy, NHSO₂C1-3 fluoroalkyl, (CO)C1-3 fluoroalkyl, (CO)C1-3 fluoroalkoxy, (CO)NHC1-3 fluoroalkyl, (CO)N(C1-3 alkyl)C1-3 fluoroalkyl, (CO)N(C4-6 fluoroalkylene), SO₂NHC1-3 fluoroalkyl, preferably H or fluor,
wherein at least one Ri or Rk comprise at least one detectable label selected in the group consisting of labelled halogen, such as ¹³¹I, ¹²³I, ¹²⁴I, ¹²⁵I, ⁷⁶B, ⁷⁵Br, ¹⁸F, ¹⁹F, ¹¹C, ¹³C, ¹⁴C, ⁹⁹Tc and ³H, preferably fluoro ¹⁸F.

According to an embodiment, one of Rj or Rk comprises a chelating group which may be complexed by at least one detectable label, such as technicium Tc. Chelating groups are well known in the art (such as DTPA, DOTA, DO3A and their numerous derivatives known).

According to another object, the present application describes compounds of formula (I′):

(A)-(CH═CH)ₙ—(B),

wherein A, B and n are as defined above.

Preferably, in compounds of formula (I′), B is a 6 membered cycle (B1) wherein m is 1.

According to another object, the present application describes compounds of formula (I″):

(A)-(C≡C)ₙ—(B),

wherein A, B and n are as defined above.

Preferably, in compounds of formula (I″), B is a 6 membered cycle (B1) wherein m is 1.

According to another object, the present invention also concerns the process of preparation of the compounds of the invention.

The compound and process of the present invention may be prepared in a number of ways well-known to those skilled in the art. The compounds can be synthesized, for example, by application or adaptation of the methods described below, or variations thereon as appreciated by the skilled artisan. The appropriate modifications and substitutions will be readily apparent and well known or readily obtainable from the scientific literature to those skilled in the art.

In particular, such methods can be found in R. C. Larock, Comprehensive Organic Transformations, Wiley-VCH Publishers, 1999.

The compounds (I) of the invention may be prepared by application or adaptation of the following routes, merely given for illustrative and non limiting purposes.

Charts 1.1 to 1.6 illustrate compounds comprising B1 cycles.

1.1) Benzothiazoles compounds

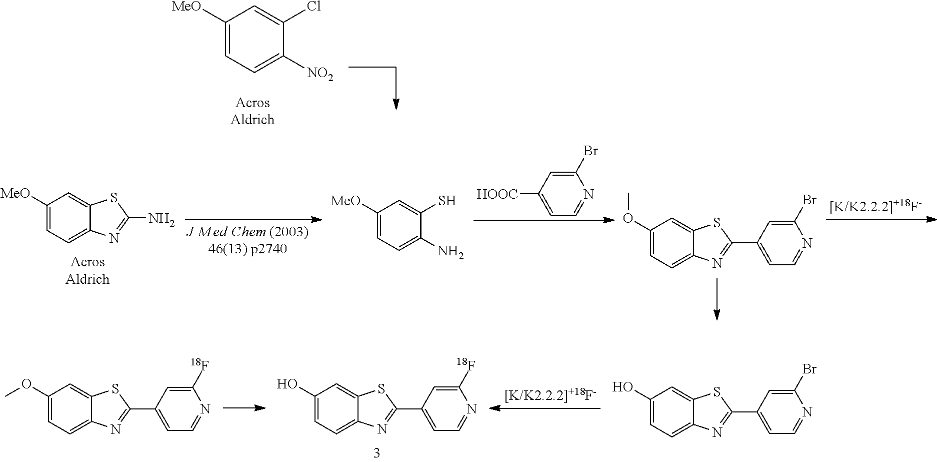

1.2) Benzoxazoles

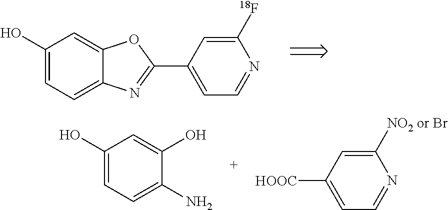

1.3) Imidazopyridines

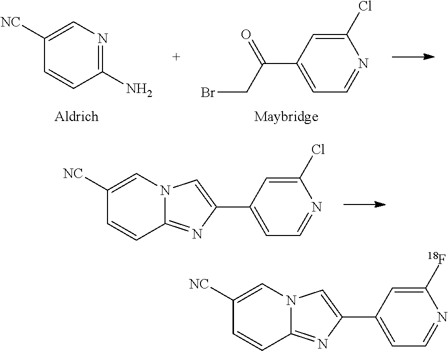

1.4) Furanes (notably Benzofuranes, diphenylfuranes)

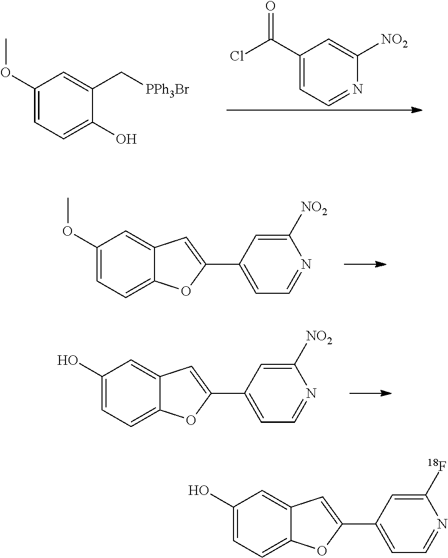

1.5) Thiophénes (notably benzothiophénes, diphenylthiophenes)

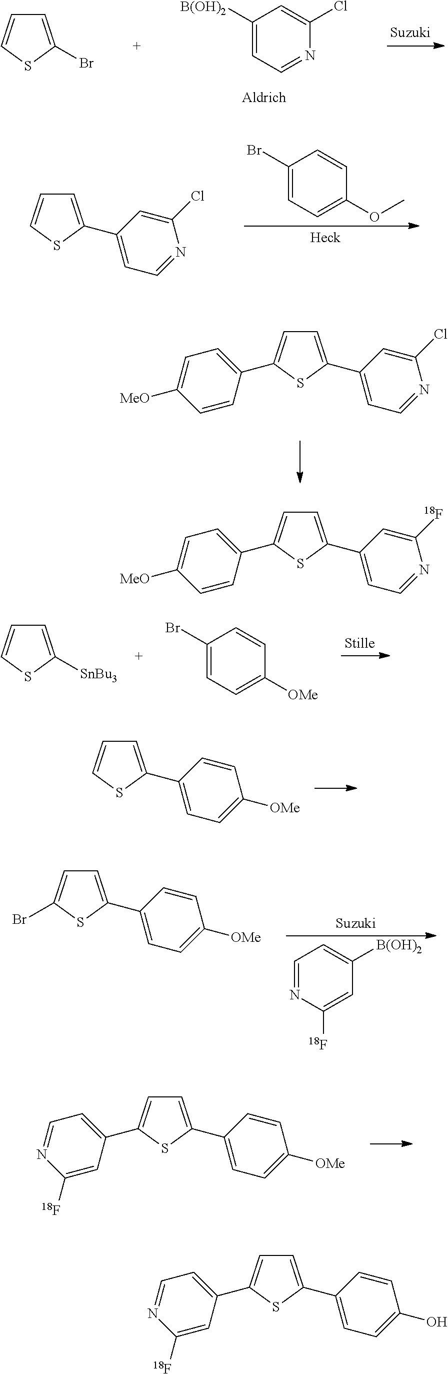

1.6) Diphenyloxadiazoles

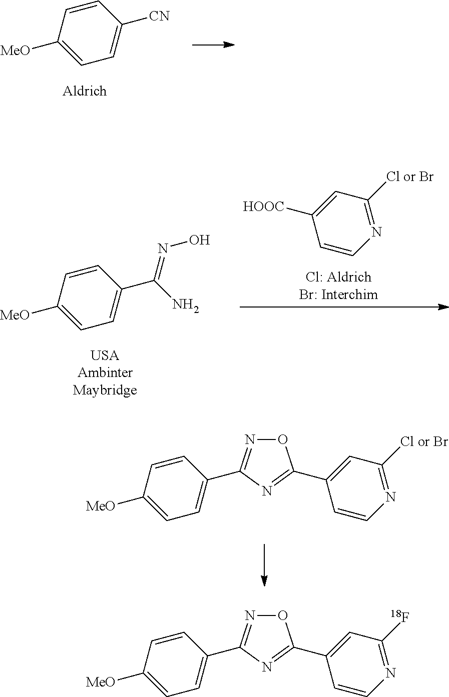

Regarding B2 five membered cycles, synthesis and radiolabeling is done in a similar approach, as illustrated in the scheme below (Charts 2.1 to 2.14). The halo intermediates obtained are converted into radiolabeled F18 compound as described in detail above for B1 six membered rings.

2.1)

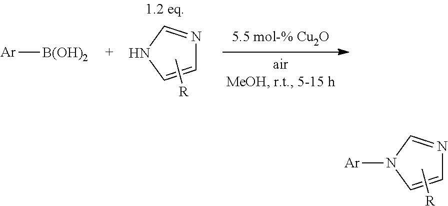

### B. Sreedhar, G. T. Venkanna, K. B. S. Kumar, V. Balasubrahmanyam, Synthesis, 2008, 795-799.

wherein Ar corresponds to a A group in formula (I), and preferably to a A1-A2 group, and R represent (Rk)ₖ groups,

2.2)

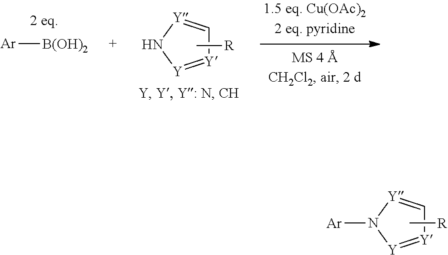

### P. Y. S. Lam, C. G. Clark, S. Saubern, J. Adams, M. P. Winters, D. M. T. Chan, A. Combs, Tetrahedron Lett., 1998, 39, 2941-2944.

wherein Ar corresponds to a A group in formula (I), and preferably to a A1-A2 group, and R represent (Rk)ₖ groups,

2.3)

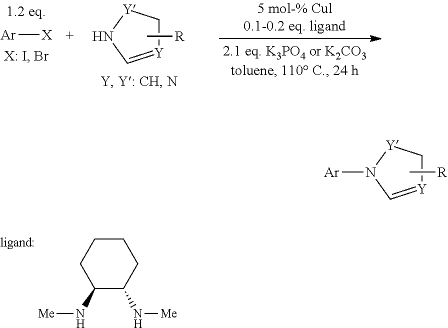

J. C. Antilla, J. M. Baskin, T. E. Barder, S. L. Buchwald, J. Org. Chem., 2004, 69, 5578-5587.
wherein Ar corresponds to a A group in formula (I), and preferably to a A1-A2 group, and R represent (Rk)ₖ groups,

2.4)

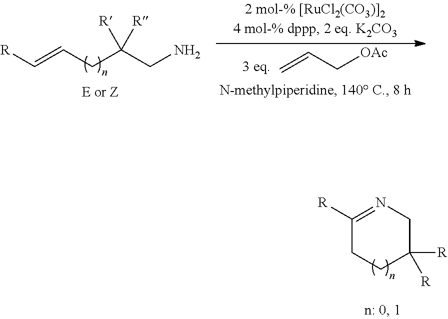

T. Kondo, T. Okada, T.-A. Mitsudo, J. Am. Chem. Soc., 2002, 124, 186-187 wherein:
R corresponds to the A-(CH2)n- group, such as A1-A2-(CH2)n-, and
R′ and R″ correspond to Rk groups.

2.5)

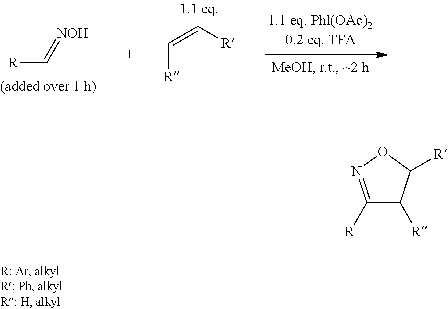

B. A. Mendelsohn, S. Lee, S. Kim, F. Tayssier, V. S. Aulakh, M. A. Ciufolini, Org. Lett., 2009, 11, 1539-1542.
wherein:
R corresponds to the A-(CH2)n- group, such as A1-A2-(CH2)n-, and
R′ and R″ correspond to Rk groups.

2.6)

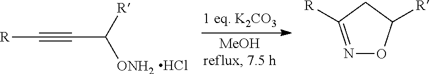

### L. Pennicott, S. Lindell, Synlett, 2006, 463-465.

wherein:
R corresponds to the A-(CH2)n- group, such as A1-A2-(CH2)n-, and
R′ and R″ correspond to Rk groups.

2.7)

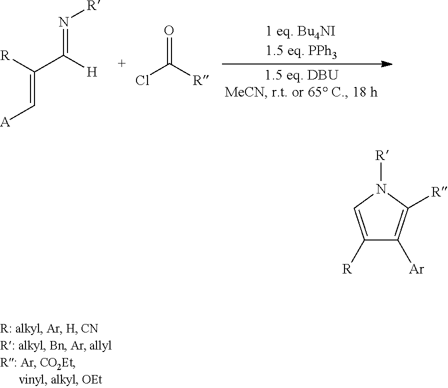

Y. Lu, B. A. Arndtsen, Org. Lett., 2009, 11, 1369-1372.
wherein Ar corresponds to the A group, for instance a fused A1-A2-(CH2)n, and R, R′, R″ correspond to Rk groups.

2.8)

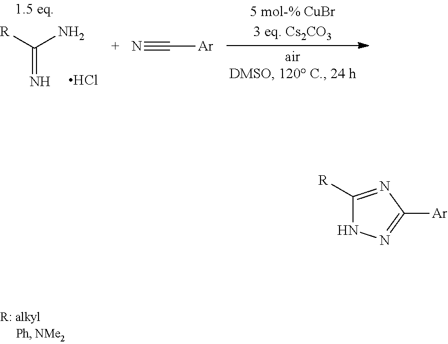

S. Ueda, H. Nagasawa, J. Am. Chem. Soc., 2009, 131, 15080-15081.

wherein Ar corresponds to the A group, for instance a fused A1-A2-(CH2)n, and R corresponds to Rk groups.

2.9)

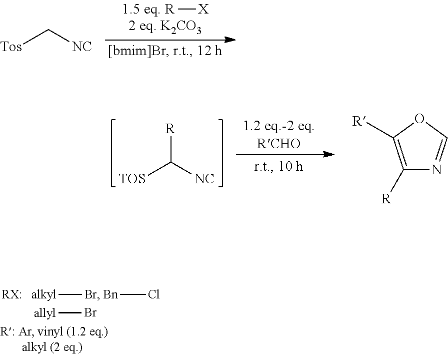

### B. Wu, J. Wen, J. Zhang, J. Li, Y.-Z. Xiang, X.-Q. Yu, Synlett, 2009, 500-504.

wherein R corresponds to the A-(CH2)n group, for instance a fused A1-A2-(CH2)n, and R′ correspond to a Rk group.

2.10)

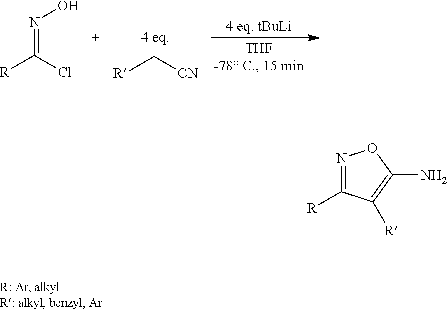

M. P. Bourbeau, J. T. Rider, Org. Lett., 2006, 8, 3679-3680.

wherein R′ corresponds to the A-(CH2)n group, for instance a fused A1-A2-(CH2)n, and R correspond to a Rk group.

2.11)

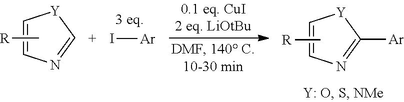

H.-Q. Do, O. Daugulis, J. Am. Chem. Soc., 2007, 129, 12404-12405.
wherein Ar corresponds to the A-(CH2)n group, for instance a fused A1-A2-(CH2)n, and R correspond to a Rk group.

2.12)

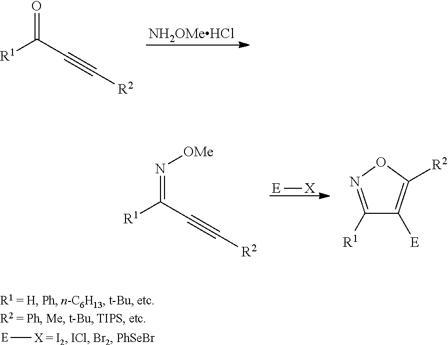

wherein either R1 or R2 corresponds to the A-(CH2)n group, for instance a fused A1-A2-(CH2)n, and the other R1 or R2 corresponds to a Rk group.

2.13) Similarly to 1.3) above, compounds wherein A is imidazopyridine are prepared by adding BrCH2CO—B2 and

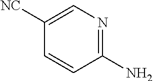

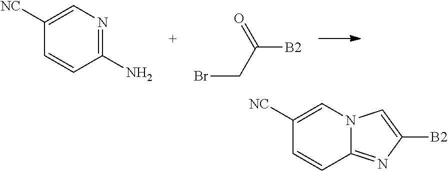

with B2 being

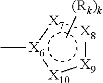

as defined in the application

2.13) Similarly to 1.1) above, compounds wherein A is benzothiazole are prepared by adding HOOC—B2 and

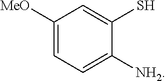

2.14) Similarly to 1.2) above, compounds wherein A is benzoxazole are prepared by adding HOOC—B2 and

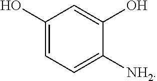

2.15) Similarly to 1.2) above, compounds wherein A is benzofurane are prepared by adding COCl-B2 and

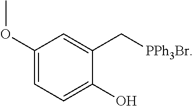

2.14)

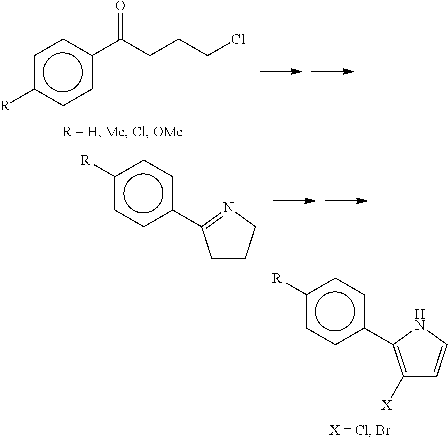

Tetrahedron, 1997, vol 53, n° 10, 3693-3706
wherein R-Ph corresponds to a A group and X corresponds to a Rk group.

It will be appreciated that the compounds of the present invention may contain one or more asymmetrically substituted carbon atoms, and may be isolated in optically active or racemic forms. Thus, all chiral, diastereomeric, racemic forms, isomeric forms of a structure are intended, unless the specific stereochemistry or isomeric form is specifically indicated. It is well-known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

Additionally, the process of the invention may lead to several regioisomers which are all encompassed by the present invention. Regioisomers are generally isolated by chromatography.

Compounds of the present invention may be prepared by a variety of synthetic routes. The reagents and starting materials are commercially available, or readily synthesized by well-known techniques by one of ordinary skill in the arts. All substituents, unless otherwise indicated, are as previously defined.

In the reactions described hereinafter, it may be necessary to protect reactive functional groups, for example hydroxyl, amino, imino, thio or carboxy groups, where these are desired in the final product, to avoid their unwanted participation in the reactions. Conventional protecting groups may be used in accordance with standard practice, for examples see T. W. Greene and P. G. M. Wuts in Protective Groups in Organic Chemistry, 3^{rd} ed., John Wiley and Sons, 1999; J. F. W. McOmie in Protective Groups in Organic Chemistry, Plenum Press, 1973.

Some reactions may be carried out in the presence of a base. There is no particular restriction on the nature of the base to be used in this reaction, and any base conventionally used in reactions of this type may equally be used here, provided that it has no adverse effect on other parts of the molecule. Examples of suitable bases include: sodium hydroxide, potassium carbonate, triethylamine, alkali metal hydrides, such as sodium hydride and potassium hydride; alkyllithium compounds, such as methyllithium and butyllithium; and alkali metal alkoxides, such as sodium methoxide and sodium ethoxide.

Usually, reactions are carried out in a suitable solvent. A variety of solvents may be used, provided that it has no adverse effect on the reaction or on the reagents involved. Examples of suitable solvents include: hydrocarbons, which may be aromatic, aliphatic or cycloaliphatic hydrocarbons, such as hexane, cyclohexane, benzene, toluene and xylene; amides, such as dimethylformamide; alcohols such as ethanol and methanol and ethers, such as diethyl ether and tetrahydrofuran.

The reactions can take place over a wide range of temperatures. In general, it is found convenient to carry out the reaction at a temperature of from 0° C. to 150° C. (more preferably from about room temperature to 100° C.). The time required for the reaction may also vary widely, depending on many factors, notably the reaction temperature and the nature of the reagents. However, provided that the reaction is effected under the preferred conditions outlined above, a period of from 3 hours to 20 hours will usually suffice.

The compound thus prepared may be recovered from the reaction mixture by conventional means. For example, the compounds may be recovered by distilling off the solvent from the reaction mixture or, if necessary, after distilling off the solvent from the reaction mixture, pouring the residue into water followed by extraction with a water-immiscible organic solvent and distilling off the solvent from the extract. Additionally, the product can, if desired, be further purified by various well-known techniques, such as recrystallization, reprecipitation or the various chromatography techniques, notably column chromatography or preparative thin layer chromatography.

In an other object of the invention, there is provided a process for preparation of a labelled compound of formula (I) from a non labelled compound of formula (I) as defined above, as synthetic precursor.

According to an aspect of the process of the invention, a labelled compound of formula (I) may be obtained from the corresponding precursor of formula (I) by reacting a radionuclide reagent.

Said radionuclide reagent may be chosen from any reagents generally used for this purpose and known from the skilled person, in particular K18F/K222, Rb18F, Cs18F, R4N+18F; more particularly, the F18 reagent is the so called K18F/K222, commercially available from Merck (Kriptofix®).

Suitable precursors generally comprise Ri, Rj, Rk groups (and preferably Rj or Rk groups of the B cycle) comprising a leaving group, a halo group (notably Br or Cl) or NO₂. For instance and preferably, the leaving group is the Rj group located at ortho position from the X3 (N atom) and Rj is Br or Cl, as shown in the detailed examples. The reaction is typically a nucleophilic substitution.

The reaction may generally be carried out in appropriate solvents such as acetonitrile, DMSO, DMF, sulfolan, dimethylacetamide. The conditions are advantageously: heating 80-180° C. for less then 30 min, or microwave activation (100W, 1-2 min).

This reaction is generally conducted quickly, as the half life of F18 is 119.8 minutes.

In situ, the radiopharmacist couples these compounds with the radionuclide produced typically by a cyclotron (for instance radioactive ¹⁸F), to make the final compound A-B labelled with the radionuclide and then ready for administration to the patient.

The precursors (I) are typically non radio-labelled derivatives corresponding to the desired labelled compound (I), designed so that the radio-labelling occurs efficiently. The precursors may also comprise an appropriate protecting group.

According to a further object of the invention, precursor compounds and methods for their preparation are also provided. Such precursors may be used as synthetic starting materials for the incorporation of labelled molecular fragments leading to radiolabelled derivatives as amyloid imaging agents. In particular, the precursors are the compounds A-(CH₂)ₙ—B not yet labelled with a radionuclide, and eventually under the form of a so-called “cold kit” sold to the radio-surgery of the hospital.

The precursor compounds of the invention, where Ri, Rj and Rk are chosen from a), b) or c) as defined above, may be obtained by application or adaptation of known methods as illustrated by the examples below. Starting compounds may be commercially available or may be obtained by application or adaptation of known compounds.

### DEFINITIONS

It is first reminded that the heterocycles mentioned as containing a C or N atom correspond respectively to the appropriate C or CH, and N or NH according to the required valence known by the one skilled in the art.

As used herein, “alkyl”, “alkylenyl” or “alkylene” used alone or as a suffix or prefix, is intended to include both branched and straight chain saturated aliphatic hydrocarbon groups having from 1 to 12 carbon atoms or if a specified number of carbon atoms is provided then that specific number would be intended. For example “C1-6 alkyl” denotes alkyl having 1, 2, 3, 4, 5 or 6 carbon atoms.

Examples of alkyl include, but are not limited to, methyl, ethyl, n-propyl, i-propyl, n-butyl, i-butyl, sec-butyl, t-butyl, pentyl, and hexyl.

Examples of alkylene or alkylenyl include, but are not limited to, C1-3 alkylene, C1-5 alkylene, methylene, ethylene, propylene, and butylene.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy. Similarly, “alkylthio” or “thioalkoxy” represent an alkyl group as defined above with the indicated number of carbon atoms attached through a sulphur bridge.

As used herein, Hal or halo, respectively fluoro, iodo, bromo, chloro represent halogen, respectively F, I, Br, Cl, including all isotopes thereof.

As used herein, “haloalkyl”, “haloalkylene” and “haloalkoxy”, used alone or as a suffix or prefix, refers to groups in which one, two, or three of the hydrogen(s) attached to the carbon(s) of the corresponding alkyl, alkylene and alkoxy-groups are replaced by halo. In particular, when halo is fluoro (notably when the compounds are radiolabelled with fluor 18), “fluoroalkyl”, “fluoroalkylene” and “fluoroalkoxy”, used alone or as a suffix or prefix, refers to groups in which one, two, or three of the hydrogen(s) attached to the carbon(s) of the corresponding alkyl, alkylene and alkoxy-groups are replaced by fluoro.

Examples of fluoroalkyl include, but are not limited to, trifluoromethyl, difluoromethyl, fluoromethyl, 2,2,2-trifluoroethyl, 2-fluoroethyl and 3-fluoropropyl. Examples of fluoroalkylene include, but are not limited to, difluoromethylene, fluoromethylene, 2,2-difluorobutylene and 2,2,3-trifluorobutylene.

Examples of fluoroalkoxy include, but are not limited to, trifluoromethoxy, 2,2,2-trifluoroethoxy, 3,3,3-trifluoropropoxy and 2,2-difluoropropoxy.

The term “aryl” refers to monocyclic or bicyclic aromatic groups containing from 6 to 12 carbons in the ring portion, preferably, 6-10 carbons in the ring portion such as phenyl, naphthyl.

As used herein, the terms “heterocycle” or “heterocyclic” refer to a saturated, partially unsaturated or aromatic (herein referred to as heteroaryl) stable 3 to 14, preferably 5 to 10 membered mono, bi or multicyclic rings wherein at least one member of the ring is a hetero atom. Typically, heteroatoms include, but are not limited to, oxygen, nitrogen, sulfur, selenium, and phosphorus atoms. Preferable heteroatoms are oxygen, nitrogen and sulfur.

Suitable heterocycles are also disclosed in The Handbook of Chemistry and Physics, 76^{th} Edition, CRC Press, Inc., 1995-1996, p. 2-25 to 2-26, the disclosure of which is hereby incorporated by reference.

Preferred non aromatic heterocyclic include, but are not limited to pyrrolidinyl, pyrazolidinyl, imidazolidinyl, oxiranyl, tetrahydrofuranyl, dioxolanyl, tetrahydro-pyranyl, dioxanyl, dioxolanyl, piperidyl, piperazinyl, morpholinyl, pyranyl, imidazolinyl, pyrrolinyl, pyrazolinyl, thiazolidinyl, tetrahydrothiopyranyl, dithianyl, thiomorpholinyl, dihydro-pyranyl, tetrahydropyranyl, dihydropyranyl, tetrahydro-pyridyl, dihydropyridyl, tetrahydropyrimidinyl, dihydrothiopyranyl, azepanyl, as well as the fused systems resulting from the condensation with a phenyl group.

The term “heteroaryl” refers to groups having 5 to 14 ring atoms, 6, 10 or 14 n electrons shared in a cyclic array and containing carbon atoms and 1, 2 or 3 O, N, or S heteroatoms. Examples of heteroaryl groups are thienyl, benzo[b]thienyl, naphtho[2,3-b]thienyl, thianthrenyl, furyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, carbazolyl, phenanthridinyl, acridinyl, perimidinyl, phenanthrolinyl, phenazinyl, isothiazolyl, phenothiazinyl, isoxazolyl, furazanyl and phenoxazinyl groups.

As used herein, “aromatic” refers to hydrocarbonyl groups having one or more unsaturated carbon ring(s) having aromatic characters, (e.g. 4n+2 delocalized electrons where “n” is an integer) and comprising up to about 14 carbon atoms. In addition “heteroaromatic” refers to groups having one or more unsaturated rings containing carbon and one or more heteroatoms such as nitrogen, oxygen or sulphur having aromatic character (e.g. 4n+2 delocalized electrons).

As used herein, “pharmaceutically acceptable” is employed to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, phosphoric, and the like; and the salts prepared from organic acids such as lactic, maleic, citric, benzoic, methanesulfonic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used.

As used herein “stable compound” and “stable structure” are meant to indicate a compound that is sufficiently robust to survive isolation to a useful degree of purity from a reaction mixture, and subsequent prolonged storage in the cold or at ambient temperature, and optionally formulated into an efficacious therapeutic or diagnostic agent.

Compounds of the invention further include hydrates and solvates.

The present invention includes isotopically labelled compounds of the invention. An “isotopically-labelled”, “radio-labelled”, “labelled”, “detectable” or “detectable amyloid binding” compound, or a “radioligand” is a compound of the invention where one or more atoms are replaced or substituted by an atom having an atomic mass or mass number different from the atomic mass or mass number typically found in nature (i.e., naturally occurring), and in particular by a radionuclide such as F18 or C11.

Further details of the corresponding definitions are know by the one skilled in the art, reminded notably in WO2008/108729, and are incorporated by reference.

The radionuclide that is incorporated in the instant radiolabelled compounds will depend on the specific application of that radiolabelled compound. For in vivo imaging applications 11C, 13C, 18F, 19F, 120I, 123I, 131I, 75Br, or 76Br will generally be most useful, in particular F18.

Examples of an “effective amount” include amounts that enable imaging of amyloid deposit(s) in vivo, that yield acceptable toxicity and bioavailability levels for pharmaceutical use, and/or prevent cell degeneration and toxicity associated with fibril formation.

Further details of method of use of the compounds of the applicant (doses, protocols of administration . . . ) are also know by the one skilled in the art, reminded notably in WO2008/108729 and are incorporated by reference.

The compounds of the present invention may be used to determine the presence, location and/or amount of one or more amyloid deposit(s) in an organ or body area, including the brain, of an animal or human. Amyloid deposit(s) include, without limitation, deposit(s) of A[β]. In allowing the temporal sequence of amyloid deposition to be followed, the compounds of the invention may also be used to correlate amyloid deposition with the onset of clinical symptoms associated with a disease, disorder or condition. The inventive compounds may be used to prevent, treat and/or to diagnose a disease, disorder or condition characterized by amyloid deposition, such as AD. Compounds of formula (I) have several potential targets including NFTs and SPs, relating to Ab plaques and/or Tau aggregates, useful notably for the early diagnostic of AD.

The method of the invention determines the presence and location of amyloid deposits in an organ or body area, preferably brain, of a patient. The present method comprises administration of a detectable quantity of a pharmaceutical composition containing an amyloid-binding compound of the present invention called a “detectable compound,” or a pharmaceutically acceptable water-soluble salt thereof, to a patient. A “detectable quantity” means that the amount of the detectable compound that is administered is sufficient to enable detection of binding of the compound to amyloid. An “imaging effective quantity” means that the amount of the detectable compound that is administered is sufficient to enable imaging of binding of the compound to amyloid.

The invention employs amyloid probes which, in conjunction with non-invasive neuroimaging techniques such as gamma imaging such as positron emission tomography (PET) or single-photon emission computed tomography (SPECT), and eventually MRI, are used to quantify amyloid deposition in viva

In addition to a high affinity for amyloid b plaques, the preferred tracer agents of the invention may exhibit several features:
they are preferably able to cross the blood-brain barrier (BBB). This requires advantageously a neutral molecule with a molecular mass not exceeding 600;
the log octanol-buffer partition coefficient, which is a measure of the lipophilicity of the compound, should be preferably between 1 and 3.

For purposes of in vivo imaging, the type of detection instrument available is a major factor in selecting a given label. For instance, isotopes such as ¹⁸F and ¹⁹F are particularly suitable for in vivo imaging in the methods of the present invention. The type of instrument used will guide the selection of the radionuclide or stable isotope. For instance, the radionuclide chosen must have a type of decay detectable by a given type of instrument. Another consideration relates to the half-life of the radionuclide. The half-life should be long enough so that it is still detectable at the time of maximum uptake by the target, but short enough so that the host does not sustain deleterious radiation. The radiolabelled compounds of the invention can be detected using gamma imaging wherein emitted gamma irradiation of the appropriate wavelength is detected. Methods of gamma imaging include, but are not limited to, SPECT and PET. Preferably, for SPECT detection, the chosen radiolabel will lack a particulate emission, but will produce a large number of photons in a 140-200 keV range.

For PET detection, the radiolabel will be a positron-emitting radionuclide, such as ¹¹C, ¹⁸F, which will annihilate to form two gamma rays which will be detected by the PET camera.

The compounds of the present invention may be administered by any means known to one of ordinary skill in the art. For example, administration to the subject may be local or systemic and accomplished orally, parenterally, by inhalation spray, topically, rectally, nasally, buccally, vaginally, or via an implanted reservoir. The term “parenteral” as used herein includes subcutaneous, intravenous, intraarterial, intramuscular, intraperitoneal, intrathecal, intraventricular, intrasternal, intracranial, and intraosseous injection and infusion techniques. The exact administration protocol will vary depending upon various factors including the age, body weight, general health, sex and diet of the patient; the determination of specific administration procedures would be routine to any one of ordinary skill in the art.

According to a further object, the present invention further provides a pharmaceutical composition, in particular for in vivo imaging of amyloid deposits, comprising a labelled compound (I) (in an effective amount) together with a pharmaceutically acceptable carrier.

The composition may comprise one or more additional pharmaceutically acceptable ingredient(s), including without limitation one or more wetting agent(s), buffering agent(s), suspending agent(s), lubricating agent(s), emulsifier(s), disintegrant(s), absorbent(s), preservative(s), surfactant(s), colorant(s), flavorant(s), sweetener(s) and therapeutic agent(s). In one embodiment, the composition is formulated for intravenous administration and the carrier includes a fluid and/or a nutrient replenisher.

In another embodiment, the composition is capable of binding specifically to amyloid in vivo, is capable of crossing the blood-brain barrier, is non-toxic at appropriate dose levels and/or has a satisfactory duration of effect.

In yet another embodiment, the composition comprises about 10 mg of human serum albumin and from about 0.0005 to 500 mg of a compound (I) of the present invention per mL of phosphate buffer containing NaCl.

Dose levels on the order of about 0.001 μg/kg/day to about 10,000 mg/kg/day of an inventive compound are useful for the inventive methods. In one embodiment, the dose level is about 0.001 μg/kg/day to about 10 g/kg/day. In another embodiment, the dose level is about 0.01 μg/kg/day to about 1.0 g/kg/day. In yet another embodiment, the dose level is about 0.1 mg/kg/day to about 100 mg/kg/day.

Any known administration regimen for regulating the timing and sequence of drug delivery may be used and repeated as necessary to effect treatment in the inventive methods. The regimen may include pretreatment and/or co-administration with additional therapeutic agent(s).

In one embodiment, the compounds (I) are administered to a subject that is suspected of having or that is at risk of developing a disease, disorder or condition characterized by amyloid deposition. Typically, the subject may be an elderly human.

The present invention further provides methods of diagnosing, treating or preventing an Aβ-related pathology in a patient, comprising administering to the patient a therapeutically effective amount of a labelled compound of formula (I).

The present invention further provides a labelled compound of formula (I) described herein for diagnosing, treating or preventing an Aβ-related pathology. The present invention further provides a compound of formula (I) described herein for the manufacture of a medicament, in particular a diagnostic contrast agent.

The present invention further provides:
a method of treating or preventing an A[β]-related pathology in a patient, comprising administering to the patient a therapeutically effective amount of a labelled compound of formula (I);
the use of a pharmaceutical composition of the invention for in vivo imaging carried out by the group of techniques selected from gamma imaging, magnetic resonance imaging and magnetic resonance spectroscopy, preferably PET imaging;
the use of a labelled compound (I) in the manufacture of a medicament for diagnostic, prevention and/or treatment of Alzheimer's disease;
the use of a labelled compound (I) for determining the efficacy of therapy in the treatment of Alzheimer's disease;
a labelled compound (I) described herein for use as a medicament.

In the present application, the terms “ARA-related pathology” or “Alzheimer's disease” or “amyloidosis” refers in particular to Alzheimer's disease and known related diseases which comprise Downs syndrome, a RA-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In still another embodiment, there is provided a method of identifying a patient as prodromal to a disease associated with amyloid deposition comprising:

(A) administering to the patient, who is presenting with signs of clinical dementia or clinical signs of a mild cognitive impairment, an amyloid binding labelled compound of formula (I) or a pharmaceutically acceptable salt thereof; then
(B) imaging said patient to obtain data; and
(C) analyzing said data to ascertain amyloid levels in said patient with reference to a normative level, thereby identifying said patient as prodromal to a disease associated with amyloid deposition.

In still another embodiment, alone or in combination with any other embodiment herein described, the invention provides the use of a labelled compound according to formula (I), as herein defined, for determining the efficacy of therapy in the treatment of amyloidosis. Another embodiment is the use of a formula (I) labelled compound in the preparation of a medicament for determining the efficacy of therapy in the treatment of amyloidosis. The method comprises typically:

(A) administering to a patient in need thereof an effective amount of an amyloid binding labelled compound of formula (I) or a pharmaceutically acceptable salt thereof;

(B) imaging said patient; then

(C) administering to said patient in need thereof at least one anti-amyloid agent;

(D) subsequently administering to said patient in need thereof an effective amount of a labelled compound of formula (I);

(E) imaging said patient; and

(F) comparing levels of amyloid deposition in said patient before treatment with said at least one anti-amyloid agent to levels of amyloid deposition in said patient after treatment with said at least one anti-amyloid agent.

Some compounds of formula (I) may have stereogenic centres and/or geometric isomeric centres (E- and Z-isomers), and it is to be understood that the invention encompasses all such optical isomers, enantiomers, diastereoisomers, atropisomers and geometric isomers.

The present invention relates to the compounds of formula (I) as hereinbefore defined as well as to the salts thereof. Salts for use in pharmaceutical compositions will be pharmaceutically acceptable salts, but other salts may be useful in the production of the compounds of formula (I).

Compounds of the invention can be used as medicaments. In some embodiments, the present invention provides compounds of formula (I), or pharmaceutically acceptable salts, tautomers thereof, for use as medicaments.

Further, the labelled compounds (I) may be administered in combination, at the same time or at a differed time with other imaging or therapeutic agents targeting Alzheimer's disease. For instance they can be used before or after a MRI contrast agent, notably a nanoparticle such as an iron oxyde nanoparticle (eventually coated with biovectors such as peptides and/or with PEG groups or aminoalcool groups) that are able to target amyloid plaques or inflammatory zones associated to amyloid plaques. For instance the MRI agent is administered first and the PET imaging agent (I) is injected afterwards or the other way round. The compound and eventually other diagnostic agents may be administered in different areas of the brain presumed to be linked to the same or to a different stage of the disease. Any appropriate mapping of the disease may be advantageously constructed. Different known imaging modalities for Alzheimer's disease such as cerebral blood volume methodologies may be also used.

It is reminded that for the radiolabelling, the one skilled in the art is also aware of techniques described notably in the following documents and references quoted in the present application:
Klunk, W. E. and Mathis, C. A. Jr (2007) Preparation of isotopically labelled benzothiazole compounds as imaging agents for amyloidogenic proteins.
Mason, N. S., et al. (2007) Synthesis and evaluation of [18F]-PIB analogs as A beta plaque PET imaging agents. J Label Compd Radiopharm. 50(Suppl 1), S87.
Stephenson, K. A. et al. (2007) Fluoro-pegylated (FPEG) imaging agents targeting Abeta aggregates. Bioconjug Chem. 18(1), 238-46.
Cai, L. et al. (2004) Synthesis and evaluation of two 18F-labelled 6-iodo-2-(4′-N,N-dimethylamino)phenylimidazo[1,2-a]pyridine derivatives as prospective radioligands for beta-amyloid in Alzheimer's disease. J Med. Chem. 47(9), 2208-18.

The following examples of synthesis of A-B1 compounds are given for illustrative, non-limiting purposes (see 1.1) to 1.4)). Similar protocols may be used for the preparation of A-B2 compounds, as illustrated in 1.7) below.

1) Chemical Synthesis

The compounds (I) of the invention may be prepared according to anyone of the following routes:

1.1) Benzothiazoles Compounds with a B1 Group,

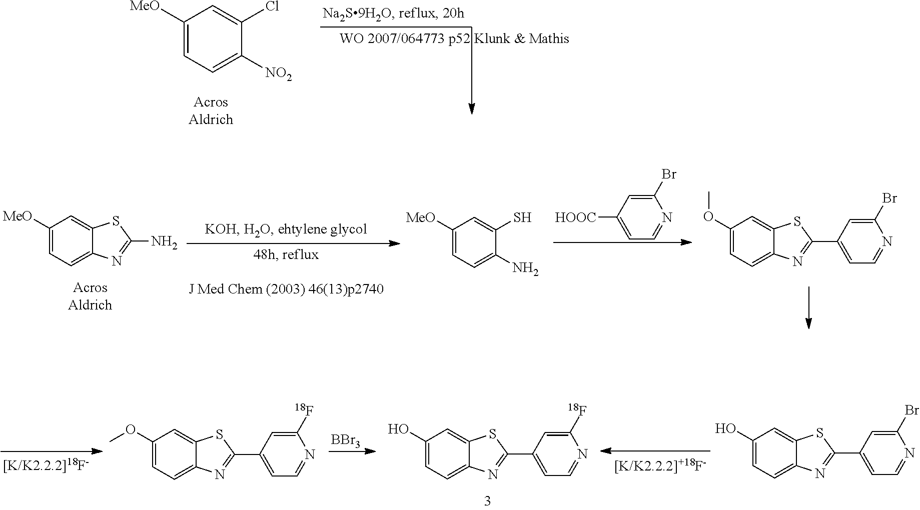

Referring to the general description the labelled compound (I) of formula 3 obtained is:

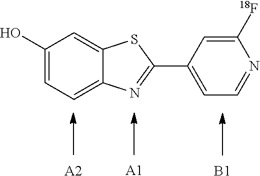

It may be obtained according to the following protocol:

### 2-Amino-5-methoxythiophenol

2-Amino-6-methoxy-benzothiazole (10 g, 57 mmol) was suspended in 50% KOH (60 g KOH dissolved in 60 mL water) and ethylene glycol (15 mL). The suspension was heated to reflux for 48 h. Upon cooling to room temperature, toluene (100 mL) was added and the reaction mixture was neutralized with acetic acid (60 mL). The organic layer was separated, and the aqueous layer was extracted with toluene. The toluene layers were combined and washed with water and dried over MgSO₄. Evaporation of the solvent gave 5 g of 2-amino-5-methoxythiophenol as yellow solid. 1H NMR (300 MHz, DMSO-d6) δ: 6.72 (d, 1H), 6.54 (d, 1H), 6.37 (dd, 1 H), 3.85 (s, 3H); MS (ES) m/z (M+H) 156.4

### 2-(2-Bromopyridin-4-yl)-6-methoxy-1,3-benzothiazole

2-bromo-4-pyridinecarboxylic acid (1.9 g, 12.7 mmol) and 2-amino-5-methoxythiophenol (2 g, 13.3 mmol) were mixed together with PPA (5 g) and heated to 170° C. under N₂ atmosphere for 2 h. The reaction mixture was cooled to room temperature and poured into 10% K₂CO₃ solution. The precipitate was filtered under reduced pressure. The crude product was purified by flash column chromatography (0 to 2% methanol in DCM) to give the 2-(2-Bromopyridin-4-yl)-6-methoxy-1,3-benzothiazole (1.1 g). 1H NMR (300 MHz, DMSO-d6) δ: 8.92 (d, 1H) 8.63 (d, 1H) 7.95 (d, 1H) 7.76 (d, 1H) 7.42 (dd, 1H) 7.11 (dd, 1H) 3.89 (s, 3H); MS (ES) m/z (M+H) 322.

### 2-(2-Bromopyridin-4-yl)-6-hydroxy-1,3-benzothiazole

To a solution of 2-(2-Bromopyridin-4-yl)-6-methoxy-1,3-benzothiazole (500 mg) in DCM (anhydrous, 10 mL) was added BBr₃ (20 mL, 1.0 M DCM solution) was injected at 0° C. under N₂ atmosphere. The reaction mixture was allowed to warm slowly and stirred at room temperature overnight. After water was added, the reaction mixture was stirred for another 0.5 h. The organic layer was separated, and the aqueous layer was extracted with DCM. The combined organic layers were dried over MgSO₄, evaporated in vacuo, and the crude product was purified by prep. HPLC to give 2-(2-Bromopyridin-4-yl)-6-hydroxy-1,3-benzothiazole (250 mg). 1H NMR (300 MHz, DMSO) δ: 9.65 (s, 1H) 8.95 (d, 1H) 8.65 (d, 1H) 7.83 (d, 1H) 7.51 (d, 1H) 7.31 (dd, 1H) 7.18 (dd, 1H); MS (ES) m/z (M+H) 308.2.

### 2-(2-[¹⁸F]Fluoropyridin-4-yl)-6-hydroxy-1,3-benzothiazole. [18F]Fluoride, produced by a cyclotron using 18O(p,n)18F reaction, was passed through a Sep-Pak Light QMA cartridge as an aqueous solution in [18]O-enriched water. The cartridge was dried by airflow, and the 18F activity was eluted with 2 ml of Kryptofix 222 (K222)/K2CO3 solution (28 mg of K222 and 2.5 mg of K2CO3 in 0.75 ml CH3CN/H2O 5:95 v/v). The solvent was removed at 120° C. for 7 min under an argon stream. The residue was azeotropically dried with 1 ml of anhydrous CH3CN twice at 120° C. under an argon stream.

A solution of bromo precursor (4 mg) in DMF (0.5 ml) was added to the reaction vessel containing the dried 18F activities. The solution was heated at 100° C. for 15 min in a closed vial to provide the crude radiolabeled compound.

The mixture was cooled down to room temperature and after dilution with an equal volume of 0.05 M ammonium acetate purified with RP-HPLC using an XTerra Prep RP18 10 mm×250 mm column (Waters) eluted isocratically with a mixture of 50% 0.05 M NH4OAc and 50% ethanol/tetrahydrofuran (75:25 v/v) at a flow rate of 3 mL/min. The fraction containing the isolated radioactive compound was diluted with an equal volume of water and then applied on an activated Sep-Pak_Plus C18 cartridge (Waters) that was rinsed with 10 ml water and then eluted with 1 ml ethanol. The purity of the labeled tracers was analyzed using an XTerra RP18 5 μm, 4.6 mm×250 mm column (Waters) eluted with an isocratic mixture of 50% 0.05 M NH4OAc and 50% ethanol/tetrahydrofuran (75:25 v/v) at a flow rate of 1 mL/min (Rt [18F]Cpd=28.5 min)

The preparation took 90 min and the radiochemical yield was 20% (decay corrected). The average specific activity was found to be 105 GBq/μmol at end of synthesis.

A similar protocol is used for the structures 1.2 to 1.6 below.

1.2) Benzoxazoles

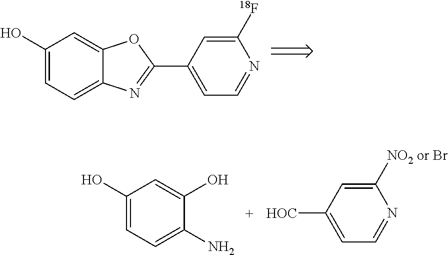

### 2-(2-Nitropyridin-4-yl)-6-hydroxy-1,3-benzoxazole

Resorcinol hydrochloride (300 mg, 1.86 mmol) was suspended in acetonitrile (5 ml) and TEA (0.5 ml) was added; the mixture was stirred for an hour. Yb(OTf)₃ (5 mg) and 2-nitro-pyridine-4-carbaldehyde (220 mg, 1.6 mmol) were then introduced portionwise over 2 h. The reaction mixture was stirred for an additional 2 h before DDQ (336 mg, 1.86 mmol) was added; stirring at room temperature was maintained overnight. Solvent was evaporated in vacuo and the crude residue was eluted through a silica gel column (95/5 DCM/MeOH) to give the 2-(2-nitropyridin-4-yl)-6-hydroxy-1,3-benzoxazole (170 mg). 1H NMR (300 MHz, DMSO-d6) 9.72 (s, 1H) 8.96 (d, 1H) 8.14 (d, 1H) 7.57 (dd, 1H) 7.45 (d, 1H) 7.02 (d, 1H) 6.78 (dd, 1H); MS (ES) m/z (M+H) 258.2

### 2-(2-[¹⁸F]Fluoropyridin-4-yl)-6-hydroxy-1,3-benzoxazole

2-(2-[¹⁸F]fluoropyridin-4-yl)-6-hydroxy-1,3-benzoxazole was radiolabelled from 2-(2-nitropyridin-4-yl)-6-hydroxy-1,3-benzoxazole precusor as same procedure as radiolabelling of 2-(2-[¹⁸F]fluoropyridin-4-yl)-6-hydroxy-1,3-benzothiazole.

1.3) Imidazopyridines

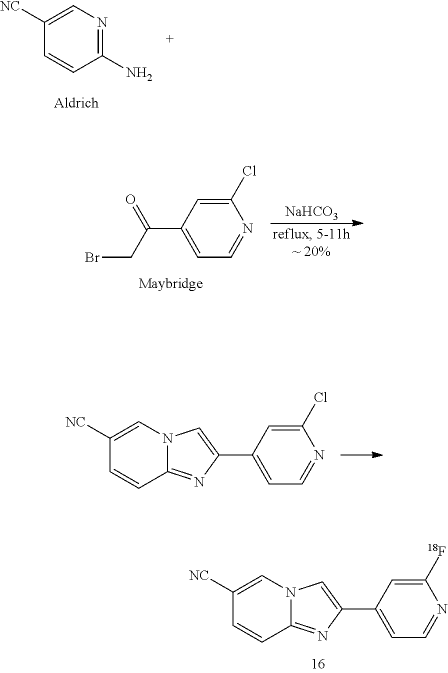

### 2-(2-Chloropyridin-4-yl)-6-cyano-imidazo[1,2-a]pyridine

6-Aminonicotinonitrile (2 g, 16.8 mmol) and 2-bromo-1-(2-chloro-4-yl)-ethanone (3.9 g, 16.8 mmol) were refluxed in ethanol (100 ml) for 2 h. Pale-yellow precipitate formed. NaHCO₃ (2 g) was added to the cooled reaction mixture and the mixture was refluxed for another 4 h. After cooling, the precipitate was filtrated, washed with water and recrystallized from ethyl acetate to give the 2-(2-chloropyridin-4-yl)-6-cyano-imidazo[1,2-a]pyridine (1.7 g). 1H NMR (300 MHz, DMSO-d6) δ: 8.70 (d, 1H) 8.30 (d, 1H) 7.63 (s, 1H) 7.35 (d, 1H) 7.29 (d, 1H) 7.21 (dd, 1H) 7.14 (dd, 1H); MS (ES) m/z (M+H) 255.7

### 2-(2-[¹⁸F]Fluoropyridin-4-yl)-6-cyano-imidazo[1,2-a]pyridine

2-(2-[¹⁸F]fluoropyridin-4-yl)-6-cyano-imidazo[1,2-a]pyridine was radiolabelled from 2-(2-chloropyridin-4-yl)-6-cyano-imidazo[1,2-a]pyridine precusor as same procedure as radiolabelling of 2-(2-[¹⁸F]fluoropyridin-4-yl)-6-hydroxy-1,3-benzothiazole.

1.4) Furanes (Notably Benzofuranes, Diphenylfuranes)

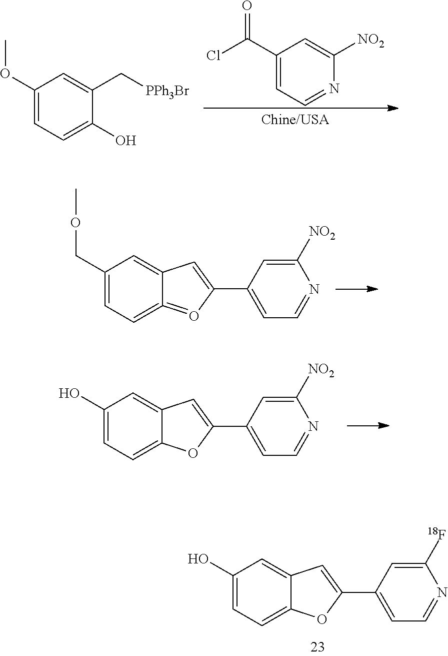

### 2-(2-Nitropyridin-4-yl)-6-methoxy-benzofuran

A mixture of 2-hydroxy-4-methoxybenzyl-triphenylphosphonium bromide (1 g, 6.5 mmol) and 2-nitro-isonicotinoyl chloride (1.21 g, 6.5 mmol) in a mixed solvent (toluene 30 ml and TEA 1 ml) was stirred under reflux for 2 h. The precipitate was removed by filtration. The filtrate was concentrated, and the residue was recrystallized from ethyl acetate to give the 2-(2-nitropyridin-4-yl)-6-methoxy-benzofuran (544 mg). 1H NMR (300 MHz, DMSO-d6) δ: 8.66 (d, 1H) 8.02 (d, 1H) 7.58 (m, 2H) 7.45 (d, 1H) 7.02 (d, 1H) 6.78 (dd, 1H) 3.83 (s, 3H); MS (ES) m/z (M+H) 271.2

### 2-(2-Nitropyridin-4-yl)-6-hydroxy-benzofuran

BBr₃ (9.4 ml, 1M solution in DCM) was added dropwise to a solution of 2-(2-nitropyridin-4-yl)-6-methoxy-benzofuran (500 mg, 1.85 mmol) in DCM (20 ml) in an ice bath. The mixture was allowed to warm to room temperature and stirred for 30 min. Water (40 ml) was added while the reaction mixture was cooled in an ice bath. The mixture was extracted with ethyl acetate, and the organic phase was dried over MgSO₄ and filtered. The filtrate was concentrated, and the residue was eluted through a C18 column (1/1 acetonitrile/H₂O) to give the 2-(2-nitropyridin-4-yl)-6-hydroxy-benzofuran (71.1 mg). 1H NMR (300 MHz, DMSO-d6) δ: 9.78 (s, 1H) 8.63 (d, 1H) 8.05 (d, 1H) 7.55 (m, 2H) 7.43 (d, 1H) 7.02 (d, 1H) 6.73 (dd, 1H); MS (ES) m/z (M+H) 257.2

### 2-(2-[¹⁸F]Fluoropyridin-4-yl)-6-hydroxy-benzofuran

2-(2-[¹⁸F]Fluoropyridin-4-yl)-6-hydroxy-benzofuran was radiolabelled from 2-(2-nitropyridin-4-yl)-6-hydroxy-benzofuran precusor as same procedure as radiolabelling of 2-(2-[¹⁸F]fluoropyridin-4-yl)-6-hydroxy-1,3-benzothiazole.

1.5) Thiophenes (Notably Benzothiophenes, Diphenylthiophenes)

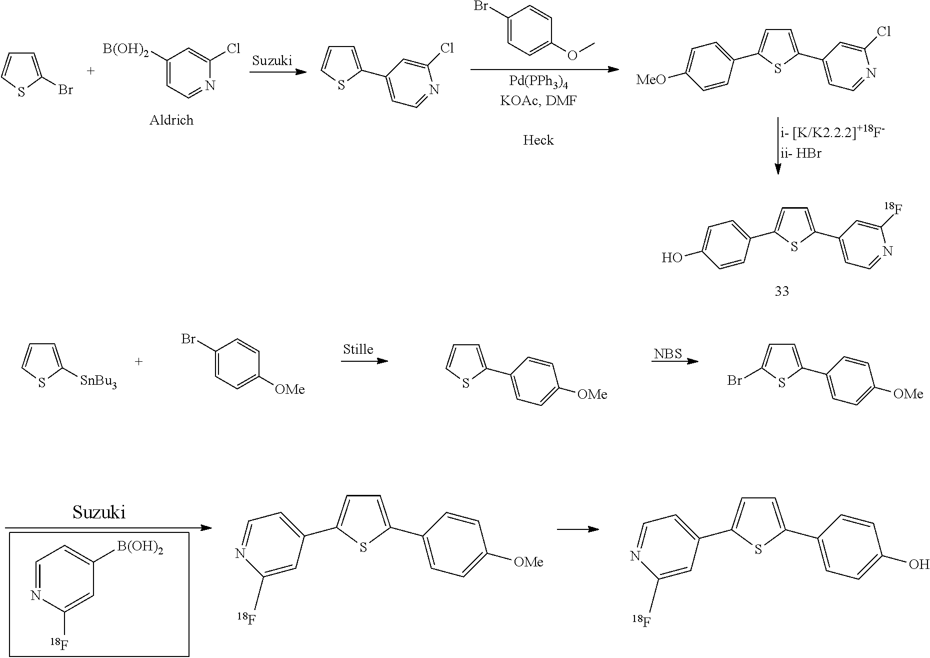

### 2-(2-Nitropyridin-4-yl)-6-methoxy-benzothiophene

2-(2-Nitropyridin-4-yl)-6-methoxy-benzothiophene (726 mg) was synthesized from 2-sulfanyl-4-methoxybenzyl-triphenylphosphonium bromide (1 g, 5.9 mmol) and 2-nitro-isonicotinoyl chloride (1.1 g, 5.9 mmol) precursors as same procedure as preparation of 2-(2-nitropyridin-4-yl)-6-methoxy-benzofuran. 1H NMR (300 MHz, DMSO-d6) δ: 8.66 (d, 1H) 8.02 (d, 1H) 7.55 (m, 2H) 7.46 (d, 1H) 7.08 (d, 1H) 6.78 (dd, 1H) 3.84 (s, 3H); MS (ES) m/z (M+H) 287.3

### 2-(2-Nitropyridin-4-yl)-6-hydroxy-benzothiophene

2-(2-Nitropyridin-4-yl)-6-hydroxy-benzo thiophene (185 mg) was synthesized from 2-(2-Nitropyridin-4-yl)-6-methoxy-benzothiophene (500 mg, 1.75 mmol) precursor as same procedure as preparation of 2-(2-nitropyridin-4-yl)-6-hydroxy-benzofuran. 1H NMR (300 MHz, DMSO-d6) δ: 9.76 (s, 1H) 8.61 (d, 1H) 8.08 (d, 1H) 7.51 (m, 2H) 7.43 (d, 1H) 7.02 (d, 1H) 6.77 (dd, 1H); MS (ES) m/z (M+H) 273.3

### 2-(2-[¹⁸F]Fluoropyridin-4-yl)-6-hydroxy-benzothiophene

2-(2-[¹⁸F]Fluoropyridin-4-yl)-6-hydroxy-benzothiophene was radiolabelled from 2-(2-nitropyridin-4-yl)-6-hydroxy-benzothiophene precusor as same procedure as radiolabelling of 2-(2-[¹⁸F]fluoropyridin-4-yl)-6-hydroxy-1,3-benzothiazole.

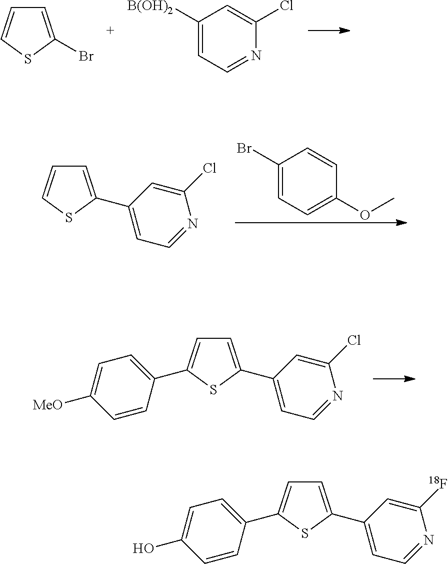

### 2-Chloro-4-thiophen-2-yl-pyridine

To a mixture of 2-bromo-thiophene (1 g, 6.1 mmol) and 2-chloropyridine-4-boronic acid (1.6 g, 10.2 mmol) in 50 mL of anhydrous DMF was added 2M Na₂CO₃ (10 ml). After degassing the mixture for 15 min, Pd(PPh₃)₄ (5 mol %) was added and the mixture was heated at 100° C. for 24 h and cooled to room temperature. The solvent was removed under reduced pressure and the residue was taken in ethyl acetate. The ethyl acetate layer was washed successively with water and brine, and dried over anhydrous MgSO₄. The crude compound, after the evaporation of the solvent, was purified by silica gel column chromatography (50% DCM in hexane) to give 2-chloro-4-thiophen-2-yl-pyridine (936 mg). 1H NMR (300 MHz, DMSO-d6) δ: 8.63 (d, 1H) 7.27 (d, 1H) 7.20 (dd, 1H) 7.15 (d, 1H) 7.09-7.05 (m, 2H); MS (ES) m/z (M+H) 196.7

### 2-Chloro-4-[5-(4-methoxy-phenyl)-thiophen-2-yl]-pyridine

To a solution of 2-chloro-4-thiophen-2-yl-pyridine (800 mg, 4.1 mmol) in DMF (10 ml) was added p-methoxy bromobenzene (1.15 g, 6.13 mmol), Pd(PPh₃)₄ (3 mol %) and potassium acetate (602 mg, 6.13 mmol). The mixture was heated at 120° C. under nitrogen overnight. The reaction mixture was poured onto ice/water and the precipitate was collected, washed with water, hexanes and ether, dried and purified by silica gel column chromatography (30% to 50% DCM in hexanes). Removal of the solvents gave 2-chloro-4-[5-(4-methoxy-phenyl)-thiophen-2-ylFpyridine (432 mg). 1H NMR (300 MHz, DMSO-d6) δ: 8.63 (d, 1H) 7.36 (dd, 2H) 7.27 (d, 1H) 7.17 (dd, 1H) 7.09-7.05 (m, 2H) 6.75 (dd, 2H) 3.82 (s, 3H); MS (ES) m/z (M+H) 302.7

### 2-Chloro-4-[5-(4-hydroxy)-thiophen-2-yl]-pyridine

2-Chloro-4-[5-(4-hydroxy)-thiophen-2-yl]-pyridine (111 mg) was synthesized from 2-chloro-4-[5-(4-methoxy)-thiophen-2-yl]-pyridine (400 mg, 1.33 mmol) precursor as same procedure as preparation of 2-(2-nitropyridin-4-yl)-6-hydroxy-benzofuran. 1H NMR (300 MHz, DMSO-d6) δ: 9.73 (s, 1H) 8.63 (d, 1H) 7.36 (dd, 2H) 7.27 (d, 1H) 7.17 (dd, 1H) 7.09-7.05 (m, 2H) 6.75 (dd, 2H); MS (ES) m/z (M+H) 273.3

### 2-[¹⁸F]Fluoro-4-[5-(4-hydroxy)-thiophen-2-yl]-pyridine

2-[¹⁸F]Fluoro-4-[5-(4-hydroxy)-thiophen-2-yl]-pyridine was radiolabelled from 2-cloro-4-[5-(4-hydroxy)-thiophen-2-yl]-pyridine precusor as same procedure as radiolabelling of 2-(2-[¹⁸F]fluoropyridin-4-yl)-6-hydroxy-1,3-benzothiazole.

1.6) Diphenyloxadiazoles

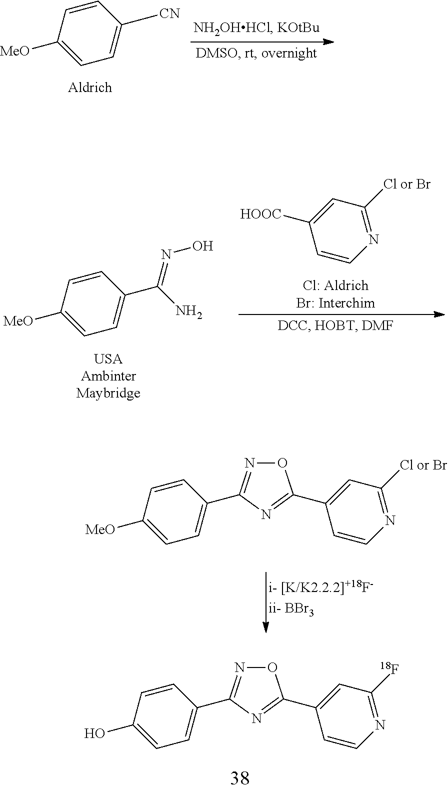

### 2-Bromo-4-[3-(4-methoxy-phenyl)-[1,2,4]oxadiazol-5-yl]-pyridine

To a stirring solution of 4-methoxybenzamidoxime (800 mg, 4.8 mmol) and 2-bromo-isonicotinic acid (972 mg, 4.8 mmol) in DMF (15 mL) was added a solution of DCC (5.8 mmol) and HOBT (9.6 mmol) in DMF (8 mL). The reaction mixture was stirred at room temperature for 20 h, and then at 100° C. for 2 additional hours. The solvent was removed, and the residue was purified by silica gel column chromatography (10% ethyl acetate in hexanes) to give 2-bromo-4-[3-(4-methoxy-phenyl)-[1,2,4]oxadiazol-5-yl]-pyridine (528 mg). ¹H NMR (300 MHz, DMSO-d6) δ: 8.61 (d, 1H) 7.33 (dd, 2H) 7.26 (d, 1H) 7.19 (dd, 1H) 6.72 (dd, 2H) 3.83 (s, 3H); MS (ES) m/z (M+H) 333.2

### 2-Bromo-4-[3-(4-hydroxy-phenyl)-[1,2,4]oxadiazol-5-yl]-pyridine

2-Bromo-4-[3-(4-hydroxy-phenyl)-[1,2,4]oxadiazol-5-yl]pyridine (156 mg) was synthesized from 2-bromo-4-[3-(4-methoxy-phenyl)-[1,2,4]oxadiazol-5-yl]-pyridine (400 mg, 1.2 mmol) precursor as same procedure as preparation of 2-(2-nitropyridin-4-yl)-6-hydroxy-benzofuran. 1H NMR (300 MHz, DMSO-d6) δ: 9.70 (s, 1H) 8.62 (d, 1H) 7.32 (dd, 2H) 7.25 (d, 1H) 7.19 (dd, 1H) 6.72 (dd, 2H); MS (ES) m/z (M+H) 318.2

### 2-[¹⁸F]Fluoro-4-[3-(4-hydroxy-phenyl)[1,2,4]oxadiazol-5-yl]-pyridine

2-[¹⁸F]Fluoro-4-[3-(4-hydroxy-phenyl)-[1,2,4]oxadiazol-5-yl]-pyridine was radiolabelled from 2-Bromo-4-[3-(4-hydroxy-phenyl)-[1,2,4]oxadiazol-5-yl]-pyridine precusor as same procedure as radiolabelling of 2-(2-[¹⁸F]fluoropyridin-4-yl)-6-hydroxy-1,3-benzothiazole.

1.7) Benzothiazoles Compounds with a B2 Group

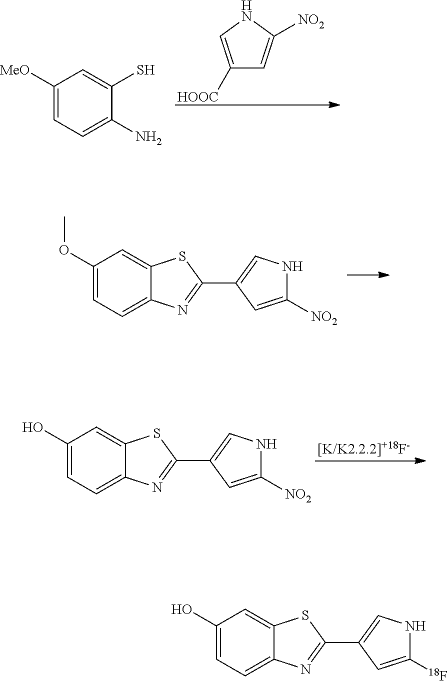

### 2-(5-Nitro-1H-pyrrol-3-yl)-6-methoxy-1,3-benzothiazole

2-(2-Nitro-1H-pyrrol-4-yl)-6-methoxy-1,3-benzothiazole was cyclizised from 5-nitro-1H-3-pyrrole-carboxylic acid (1.98 g, 12.7 mmol) and 2-amino-5-methoxythiophenol (2 g, 13.3 mmol) as same procedure as cyclization of 2-(5-Bromopyridin-4-yl)-6-methoxy-1,3-benzothiazole. 1H NMR (300 MHz, DMSO-d6) δ: 8.92 (d, 1H) 7.95 (d, 1H) 7.76 (d, 1H) 6.50 (d, 1H), 6.04 (d, 1H) 5.55 (s, 1H), 3.89 (s, 3H); MS (ES) m/z (M+H) 157.

### 2-(5-Nitro-1H-pyrrol-3-yl)-6-hydroxy-1,3-benzothiazole

2-(5-Nitro-1H-pyrrol-3-yl)-6-hydroxy-1,3-benzothiazole was demethylated from 2-(2-Nitro-1H-pyrrol-4-yl)-6-methoxy-1,3-benzothiazole as same procedure as demethylation of 2-(5-Bromopyridin-4-yl)-6-hydroxy-1,3-benzothiazole. 1H NMR (300 MHz, DMSO) δ: 9.70 (s, 1H) 8.95 (d, 1H) 7.83 (d, 1H) 7.51 (d, 1H) 6.53 (d, 1H), 6.06 (d, 1H), 5.55 (s, 1H); MS (ES) m/z (M+H) 262.

### 2-(5-[¹⁸F]Fluoro-1H-pyrrol-3-yl)-6-hydroxy-1,3-benzothiazole

2-(5-[¹⁸F]Fluoro-1H-pyrrol-3-yl)-6-hydroxy-1,3-benzothiazole was radiolabelled from 2-(5-Nitro-1H-pyrrol-3-yl)-6-hydroxy-1,3-benzothiazole precusor as same procedure as radiolabelling of 2-(5-[¹⁸F]fluoropyridin-4-yl)-6-hydroxy-1,3-benzothiazole

2) Biological Activity

The following protocol is used for testing in vitro binding of the compounds (compounds C) by using Aβ (1-42) aggregated peptides in solution.

2.1 Preparation of peptides Aβ (1-42)

The solid form of peptide Aβ (1-42) is gently dissolved at 50 μM in a sterile buffer solution (pH 7.4) containing 10 mM sodium phosphate and 1 mM EDTA. Small aliquots (200 μL) are frozen and stored at −20° C. until their use. An aliquot of peptide is thawed and incubated for 36-42 h at 37° C. under gentle and constant shaking. This incubation leads to aggregation of peptides. At the end of the incubation time, aggregated peptides are diluted in the buffer solution in order to obtain the concentrations of 20 μM. The addition of 50 μL of this solution (final volume of 1 mL) allows obtaining the testing concentrations of 1 μM.

2.2 Preparation of the Non Radioactive Ligands

A solution of test compounds is prepared at 3 mM in DMSO. These solutions are diluted in ethanol 10% in order to obtain the testing concentrations of 0.3, 1, 3, 10, 30, 100, 300 and 1000 nM.

2.3 Preparation of the Radioactive Ligand (IMPY)

The ¹²⁵I-radiolabelled ligand solution is diluted in ethanol 10% in order to obtain the final concentration of 0.05 nM in IMPY.

At the beginning of the experiment, 1 μL of ¹²⁵I-radiolabelled IMPY solution will be added to 199 μL of ethanol 10% and is counted with a gamma-counter. Fifty microliters of the working solution of ¹²⁵I-radiolabelled IMPY is added to 150 μL of ethanol 10% and is counted (background). The results obtained are compared to the theorical values and the working solution is adjusted by adding radiolabelled IMPY or ethanol 10% if needed. A variation of 10% will be accepted between theorical and experimental values.

2.4 Incubation of Aggregated Peptides with Test Compounds and ¹²⁵I-Radiolabelled IMPY.

Fifty microliters of aggregated peptides (20 μM) are incubated for 3 h under gentle agitation at room temperature with 40 μL of each non-radiolabelled test compound dilution and with 50 μL of radiolabelled ligand at 1 nM in 860 μL of ethanol 10% (quadruplicate per condition). For the total binding, 50 μL of aggregated peptides (20 μM) is incubated for 3 h under gentle agitation at room temperature with 40 μL of ethanol 10% and with 50 μL of radiolabelled ligand solution at 1 nM in 860 μL of ethanol 10% (quadruplicate per condition). Tubes (quadruplicate) without aggregated peptides are added as control of radioactivity retained on filter. Fifty microliters of peptides buffer solution are mixed with 40 μL ethanol 10% and with 50 μL of the radiolabelled ligand solution in 860 μL of ethanol 10% (quadruplicate per condition).

At the end of incubation time, the mixture is then filtered through GF/B filters by using a Brandel M24 Harvester. Filters are then washed twice with 3 mL of ethanol 10%. Filters are collected and the radioactivity is counted with a gamma-counter Cobra II.

A curve is drawn with GraphPad Prism 4.02 software in order to represent the total specific radioligand binding (cpm) as a function of the logarithm concentration of unlabelled compound. This curve allows determining the IC₅₀ for each compound. The IC₅₀ is the concentration of unlabelled test compound that blocks 50% of the specific binding. The Ki of IMPY (homologous competition) is calculated from the IC₅₀, using the following equation: Kᵢ=K_{D}=IC₅₀−L. The Ki of each compound is calculated from the IC₅₀, using the following equation: Kᵢ=IC₅₀/[1+[radioligand]/K_{D}].

2.5 Compounds Testing

Positive results and binding values (range of 1 to 5 on a [0-5] scale) indicate promising affinity towards amyloid markers (or other AD associated markers such as Tau aggregates), with values IC50 in the target range 1 to 300 nM, in particular 5 to 100 nM. Values Log P of about 2 to 4 may be obtained.

2.6 Comparaison of Compounds of Formula (I) Wherein (B) is (B1) with m=1 and the PIB Compound

The most examined compound PIB having the following formula:

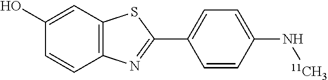

has been tested clinically and, hence demonstrated to be a potential biomarker for the visualization of Aβ plaques in AD brains with PET. However, the first generation of radioligands for PET, including the [¹¹C]PIB, are not ideal for quantification due to low signal to noise ratio, high non-specific binding or unfavourable kinetics. The non-specific binding of Aβ PET ligands should also be minimized in order to increase the sensitivity to allow for even lower levels of Aβ plaques to be detected, and thus to monitor β-amyloid lowing therapies with higher sensitivity.
Therefore, a critical need to fine-tune the binding specificity, the kinetics of the uptake and washout of Aβ radioligands derivatives exists. Previous results regarding uptake into and clearance from the brain point to high lipophilicity as one of the reasons for a slow washout from the brain and a high non-specific binding, two crucial properties for achieving an adequate signal to noise ratio.

The present inventors replaced the original phenyl group in phenyl derivatives with a less lipophilic pyridyl group (compounds of formula (I) wherein B represents (B1) with m=1) to reduce non-specific binding and increased wash-out rate of non-specific binding in vivo.

It is well known that electron-withdrawing substituents, such as a nitro or carbonyl group, present on the ortho or para position of a nitrophenyl ring, activate the nitro group and facilitate the exchange of the nitro group for a fluorine-18 atom: radiolabelling of a 4′-nitrophenyl (cycle B) derivative via a direct aromatic nucleophilic substitution with fluorine-18 would be possible as fused cycle A part could act as electron-withdrawing group. However in some cases, the radiolabeling failed because in the alkaline labelling conditions a partial negative charge is induced at position 6 (cases of amino or hydroxy substituents). As a result, the fused cycle A part no longer acts as electron-withdrawing substituent: introduction of Boc or MOM protecting groups at position 6 would allow for radiolabelling but implies an additional step of cleavage after the radiolabelling.

To overcome this problem, nucleophilic heteroaromatic substitution at the ortho-position (3′) with no-carrier-added [18F]fluoride appears as the most efficient method. Like for the aliphatic nucleophilic radiofluorinations, only a good leaving group is required (a halogen, or better a nitro- or a trimethylammonium group). There is no need for an additional strong electron-withdrawing substituent for activation of the aromatic ring such as in the homoaromatic nucleophilic radiofluorinations, except if one considers meta-fluorination. Compounds of formula (I) wherein (B) is (B1) with m is 1 can therefore be easily synthesized.

Ultimately, introduction of the nitrogen in the 4′ position of the (B1) cycle (X3=N) allows for keeping ability to form hydrogen bonds with Aβ as with the classical 4′-mono- or dimethylamino amino group: it is generally preferred that the molecules have a hydrogen bond donor both on the left-hand and right-hand side in order to specifically bind to amyloid plaques in AD brain tissue.

Additionally, the presence of the 4′ nitrogen (X3=N) instead of the mono- or dimethylamino group in PIB blocks a potential metabolic position where conversion to demethylated metabolites able to cross the blood-brain-barrier might complicate quantification of PET brain images.

### LIST OF MAIN REFERENCES

1. Hardy, J. A.; Higgins, G. A. Science 1992, 256, 184.
2. Selkoe, D. J. Physiol. Rev. 2001, 81, 741.
4. Mathis, C. A.; Wang, Y.; Klunk, W. E. Curr. Pharm. Des. 2004, 10, 1469.
8. Ono, M.; Wilson, A.; & al Nucl. Med. Biol. 2003, 30, 565.
9. Verhoeff, N.; Wilson, A.; & al Am. J. Geriatr. Psychiatr. 2004, 12, 584.
10. Agdeppa, E.; Kepe, V.; & al J. Neurosci. 2001, 21, 1.
12. Klunk, W. E.; Engler, H.; & al Ann. Neurol. 2004, 55, 306.
13. Rowe, C.; Ackerman, U.; & al Lancet Neurol. 2008, 7, 129.
14. Zhang, W.; Kung, M. P. Nucl. Med. Biol. 2007, 34, 89.
15. Mathis, C.; Lopresti, B.; & al J. Nucl. Med. 2007, 48, 56P.

1-20. (canceled)

21. Compound of formula (Ia): 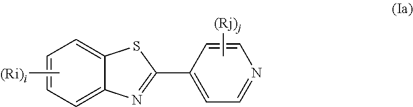
wherein:
i is from 0 to 4,
j is from 0 to 4,
for each (i) and (j), each Ri and Rj may be identical or different and are independently chosen from:
a)—H where i or j is 0,
a linear, branched or cyclic, saturated or unsaturated aliphatic group, optionally substituted by one or more of Halogen, CN, NO₂, CHalo₃, COR₃, COOR₃, CONR₃R₄, NCOR₃, NHSO₂R₃, SR₃, SOR₃, SO₂R₃, OR₃ or NR₃R₄, wherein R₃ and R₄ represents independently H or a linear, branched or cyclic alkyl group optionally substituted by one or more of Halogen;
a Halogen, CN, NO₂, CHal₃, OR₁ or NR₁R₂, COR₁, COOR₂, CONR₁R₂, NCOR₁, NHSO₂R₁, SR₁, SOR₁ or SO₂R₁ groups, wherein R₁ and R₂ represent independently H or a linear, branched or cyclic alkyl group optionally substituted by one or more of Halogen or R₁ and R₂ form together with the N atom to which they are attached a N-containing heterocycle;
or
b)—a linear, branched or cyclic, saturated or unsaturated aliphatic group, substituted by one or more of leaving group and further optionally substituted by a Halogen, CN, NO₂, CHalo₃, COR₃, COOR₃, CONR₃R₄, NCOR₃, NHSO₂R₃, SR₃, SOR₃, SO₂R₃, OR₃ or NR₃R₄, wherein R₃ and R₄ represents independently H or a linear, branched or cyclic alkyl group optionally substituted by one or more of Halogen;
a leaving group;
OR₁ or NR₁R₂, COR_{I}, COOR₂, CONR₁R₂, NCOR₁, NHSO₂R₁, SR₁, SOR₁, or SO₂R₁ groups, wherein R₁ or R₂ represent a linear, branched or cyclic alkyl group substituted by one or more of leaving group;
or
c)—a linear, branched or cyclic, saturated or unsaturated aliphatic group, substituted by one or more of Halogen and further optionally substituted by one or more of CN, NO₂, CHalo₃, COR₃, COOR₃, CONR₃R₄, NCOR₃, NHSO₂R₃, SR₃, SOR₃, SO₂R₃, OR₃ or NR₃R₄, wherein R₃ and R₄ represents independently H or a linear, branched or cyclic alkyl group optionally substituted by one or more of Halogen;
a Halogen, CHal₃,
OR₁ or NR₁R₂, COR₁, COOR₂, CONR₁R₂, NCOR₁, NHSO₂R₁, SR₁, SOR₁, or SO₂R₁ groups, wherein R₁ or R₂ represent a linear, branched or cyclic alkyl group substituted by one or more of Halogen;
or
d)—a linear, branched or cyclic, saturated or unsaturated aliphatic group, substituted by one or more of R₁₀ and further optionally substituted by one or more of Halogen, CN, NO₂, CHalo₃, COR₃, COOR₃, CONR₃R₄, NCOR₃, NHSO₂R₃, SR₃, SOR₃, SO₂R₃, OR₃ or NR₃R₄, wherein R₃ and R₄ represents independently H or a linear, branched or cyclic alkyl group optionally substituted by one or more of Halogen;
a R₁₀ group;
OR₁ or NR₁R₂, COR_{I}, COOR₂, CONR₁R₂, NCOR₁, NHSO₂R₁, SR₁, SOR₁ or SO₂R₁ groups, wherein R₁ or R₂ represent a linear, branched or cyclic alkyl group substituted by one or more of R₁₀;
wherein R₁₀ is a radionuclide, in particular selected from the group consisting of ¹²⁰I, ¹²³I, ¹²⁴I, ¹²⁵I, ¹³¹I ⁷⁵Br, ⁷⁵Br, ¹⁸F, ¹⁹F, ¹¹C, ¹³C, ¹⁴C, ⁹⁹Tc and ³H, preferably fluoro ¹³F;
provided that at least of Ri or Rj is chosen from a R₁₀ containing group d);
and their pharmaceutically acceptable salts.

22. Compound according to claim 21 wherein at least one Ri or Rj is fluoro, chloro, bromo, iodo or a fluoro containing group chosen from C1-5 fluoroalkyl, C1-3 alkyleneOC1-3 fluoroalkyl, C1-3 alkyleneNHC1-3 fluoroalkyl, C1-3 alkyleneN(C1-3 fluoroalkyl)₂, C1-3 alkyleneN(C1-3 alkyl)C1-3 fluoroalkyl, C1-5 fluoroalkoxy, C1-5 fluoroalkylthio, NHC1-3 fluoroalkyl, N(C1-3 alkyl)C1-3 fluoroalkyl, NH(CO)C1-3 fluoroalkyl, NH(CO)C1-3 fluoroalkoxy, NHSO₂C1-3 fluoroalkyl, (CO)C1-3 fluoroalkyl, (CO)C1-3 fluoroalkoxy, (CO)NHC1-3 fluoroalkyl, (CO)N(C1-3 alkyl)C1-3 fluoroalkyl, (CO)N(C4-6 fluoroalkylene)or SO₂NHC1-3 fluoroalkyl.

23. Compound according to claim 21 wherein Rj is chosen among:
chloro, fluoro, bromo, iodo, preferably fluoro; or
C1-5 fluoroalkyl, C1-3 alkyleneOC1-3 fluoroalkyl, C1-3 alkyleneNHC1-3 fluoroalkyl, C1-3 alkyleneN(C1-3 fluoroalkyl)₂, C1-3 alkyleneN(C1-3 alkyl)C1-3 fluoroalkyl, C1-5 fluoroalkoxy, C1-5 fluoroalkylthio, NHC1-3 fluoroalkyl, N(C1-3 alkyl)C1-3 fluoroalkyl, NH(CO)C1-3 fluoroalkyl, NH(CO)C1-3 fluoroalkoxy, NHSO2C1-3 fluoroalkyl, (CO)C1-3 fluoroalkyl, (CO)C1-3 fluoroalkoxy, (CO)NHC1-3 fluoroalkyl, (CO)N(C1-3 alkyl)C1-3 fluoroalkyl, (CO)N(C4-6 fluoroalkylene), SO₂NHC1-3 fluoroalkyl.

24. A compound according to claim 21 wherein at least one of Ri or Rj comprise at least one detectable label selected in the group consisting of ¹³¹I, ¹²³I, ¹²⁴I, ¹²⁵I, ¹²⁰I, ⁷⁶Br, ⁷⁵Br, ¹⁸F, ¹⁹F, ¹¹C, ¹³C, ¹⁴C, ⁹⁹Tc and ³H.

25. A compound according to claim 24 wherein Rj is ¹⁸F.

26. A compound according to claim 21 having the following formula:

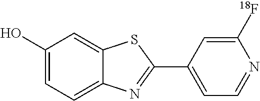

27. A pharmaceutical composition comprising a labelled compound according to claim 21 together with a pharmaceutically acceptable carrier.

28. A method of in vivo imaging of amyloid deposits, comprising administering to the patient a therapeutically effective amount of a pharmaceutical composition according to claim 27.

29. Method for in vivo imaging by a technique selected from gamma imaging, magnetic resonance imaging and magnetic resonance spectroscopy, preferably PET imaging, comprising administering to the patient a therapeutically effective amount of a compound according to claim 21.

30. Methods of diagnosing, treating or preventing an Alzheimer's disease in a patient, comprising administering to the patient a therapeutically effective amount of a compound according to claim 21.

31. A method for determining the efficacy of therapy in the treatment of Alzheimer's disease comprising administering to the patient a therapeutically effective amount of a compound according to claim 21.
